(19) United States
(12) Patent Application Publication
(10) Pub. No.: US 2008/0172183 A1
(43) Pub. Date: Jul. 17, 2008

(54) SYSTEMS AND METHODS FOR METHYLATION PREDICTION

(76) Inventors: Achim Karger, Foster City, CA (US); Victoria Boyd, San Carlos, CA (US)

Correspondence Address: MH2 TECHNOLOGY LAW GROUP, LLP, 1951 KIDWELL DRIVE, SUITE 550, TYSONS CORNER, VA, 22182, US

(21) Appl. No.: 11/673,787

(22) Filed: Feb. 12, 2007

Related U.S. Application Data
(60) Provisional application No. 60/772,264, filed on Feb. 10, 2006.

(51) Int. Cl. G06F 19/00; G01N 33/00; G06F 17/18; B01D 57/02

(52) U.S. Cl. 702/19; 436/94; 204/451; 702/179

(57) ABSTRACT

A method is provided for predicting an amount of methylation of at least one target region. The method includes establishing an observed size of a plurality of oligonucleotides relative to a size standard and correlating the observed size to a number of each of nucleotide base in each of the plurality of oligonucleotides. A mobility coefficient can be determined for each base of each respective oligonucleotide and the determined mobility coefficients can be applied to a predetermined number of polynucleotides subjected to methylation detection analysis. The plurality of oligonucleotides are treated with a modifying agent to obtain amplicons in methylated and unmethylated target regions and amplicons derived from methylated and unmethylated target regions are distinguished based on their relative mobilities. The degree of methylation can be predicted based on distinguished methylated regions.

### CROSS-REFERENCE TO COPENDING APPLICATIONS

This application claims the benefits of priority to U.S. Provisional Application No. 60/772,264, filed on Feb. 10, 2006, which is incorporated by reference in its entirety herein.

This application makes cross-reference to U.S. Provisional Application No. 60/654,162 (Client Docket No. 5692P), entitled “Compositions, Methods, and Kits for Analyzing DNA Methylation,” filed on Feb. 18, 2005, and U.S. Provisional Application No. 60/______, (Client Docket No. 5730P) entitled “Methods and Kits for Evaluating DNA Methylation,” filed on ______, both of which are also incorporated by reference herein in their entirety.

### FIELD

The present teachings generally relate to the fields of biochemistry, cell biology, and biotechnology, including systems and methods for predicting methylation of genomic DNA. Further, the present teachings include methods for predicting sizes of amplicons generated from methylated and unmethylated gDNA.

### BACKGROUND

Recently, there have been developments in determining the degree of methylation of particular genomic DNA (gDNA) target regions, as this information is invaluable in many research, diagnostic, medical, forensic, and industrial fields. The methylation of cytosine residues in gDNA is an important genetic alteration in eukaryotes. In humans and other mammals, methylcytosine is found almost exclusively in cytosine-guanine (CpG) dinucleotides. gDNA methylation plays an important role in gene regulation and changes in methylation patterns are reportedly involved in many human cancers and certain human diseases. Among the earliest and most common genetic alterations observed in human malignancies is the aberrant methylation of CpG islands, particularly CpG islands located within the 5′ regulatory regions of genes, causing alterations in the expression of such genes. Subsequently, there is great interest in using DNA methylation markers as diagnostic indicators for early detection, risk assessment, therapeutic evaluation, recurrence monitoring, and the like (see, Widschwendter et al., Clin. Cancer Res. 10:565-71, 2004; Dulaimi et al., Clin. Cancer Res. 10:1887-93, 2004; Topaloglu et al., Clin. Cancer Res. 10:2284-88, 2004; Laird, Nature Reviews, 3:253-266, 2003; Fraga et al., BioTechniques 33:632-49, 2002; Adorjan et al., Nucleic Acids Res. 30(5):e21, 2002; and Colella et al., BioTechniques, 35(1):146-150, 2003). There is also great scientific interest in the role of DNA methylation in embryogenesis, cellular differentiation, transgene expression, transcriptional regulation, and maintenance methylation, among other things.

To date, however, there has been no repeatable method for predicting migration rates of target regions in modified DNA, and further predicting sizes of components found in the target regions.

### SUMMARY

In various embodiments, the present teachings can provide a for predicting an amount of methylation of at least one target region, the method comprising establishing an observed size of a plurality of oligonucleotides relative to a size standard, correlating the observed size to the number of each of the nucleotide bases in each of the plurality of oligonucleotides, determining a mobility coefficient for each base of the respective oligonucleotide, applying determined mobility coefficients to a predetermined number of genes subjected to methylation detection analysis, treating said oligonucleotides with a modifying agent to obtain amplicons in methylated and unmethylated target regions, distinguishing amplicons derived from methylated and unmethylated target regions based on their relative mobilities, and predicting the degree of methylation of distinguished methylated regions.

In various embodiments, the present teachings can provide a method for predicting a size of amplicons generated from methylated and unmethylated gDNA, the method comprising establishing an observed size of a plurality of oligonucleotides relative to a size standard, correlating the observed size to the number of each of the nucleotide bases in each of the plurality of oligonucleotides, determining a mobility coefficient for each base of the respective oligonucleotide, applying determined mobility coefficients to a predetermined number of genes subjected to methylation detection analysis, and calculating the predicted size of the amplicons using mobility coefficients and a known sequence of the amplicon and a presumed sequence of an amplicon arising from a per-methylated gDNA.

In various embodiments, the present teachings may provide a method for calculating a predicted size for an untreated (DNA) product and a bisulfate treated (DNA) product comprising providing a known sequence of an amplicon and a presumed sequence of an amplicon arising from a per-methylated gDNA, calculating a DNA fragment size to a length of the corresponding oligonucleotide, and calculating a DNA fragment size to a composition of the corresponding oligonucleotide.

Additional embodiments are set forth in part in the description that follows, and in part will be apparent from the description, or may be learned by practice of the various embodiments described herein.

### BRIEF DESCRIPTION OF THE DRAWINGS

Various embodiments of the present teachings are exemplified in the accompanying drawings. The teachings are not limited to the embodiments depicted, and include equivalent structures and methods as set forth in the following description and known to those of ordinary skill in the art. In the drawings:

FIG. 1 illustrates a schematic representation of workflow for methylation dependent fragment separation (MDFS) according to various embodiments of the present teachings;

FIG. 2 illustrates a percentage of methylation of four amplicons examined by MDFS according to various embodiments of the present teachings;

FIG. 3 illustrates a comparison of PCR results from bisulfite-converted gDNA from a control male, control female, universally methylated male, and a fragile-X male according to various embodiments of the present teachings; and

FIG. 4 illustrates a computer system for implementing various embodiments of the present teachings.

### DESCRIPTION OF VARIOUS EMBODIMENTS

It is to be understood that both the foregoing general description and the following detailed description are exemplary and explanatory only and are not intended to limit the scope of the current teachings.

In this application, the use of the singular includes the plural unless specifically stated otherwise. Also, the use of “or” means “and/or” unless stated otherwise. Furthermore, the use of the term “including”, as well as other forms, such as “includes” and “included”, is not limiting. Also, terms such as “element” or “component” encompass both elements and components comprising one unit and elements and components that comprise more than one subunit unless specifically stated otherwise. Wherever possible, the same reference numbers will be used throughout the drawings to refer to the same or like parts.

The section headings used herein are for organizational purposes only, and are not to be construed as limiting the described subject matter in any way. All documents cited in this application, including, but not limited to patents, patent applications, articles, books, and treatises, are expressly incorporated by reference in their entirety for any purpose. In the event that one or more incorporated literature and similar materials defines or uses a term in such a way that it contradicts a term's definition in this application, this application controls. While the present teaching are described in conjunction with various embodiments, it is not intended that the present teachings be limited to such embodiments. On the contrary, the present teachings encompass various alternatives, modifications, and equivalents, as will be appreciated by those of skill in the art.

### SOME DEFINITIONS

The terms “annealing” and “hybridizing”, including variations of the root words hybridize and anneal, are used interchangeably and mean the nucleotide base-pairing interaction of one nucleic acid with another nucleic acid that results in the formation of a duplex, triplex, or other higher-ordered structure. The primary interaction is typically nucleotide base specific, e.g., A:T, A:U, and G:C, by Watson-Crick and Hoogsteen-type hydrogen bonding. In certain embodiments, base-stacking and hydrophobic interactions may also contribute to duplex stability.

The term “at least some of”, for example, when used in reference to a sample or a modified sample, means that all of the sample or modified sample can be used or that some, but not all, of the sample or modified sample can be used, for example, an aliquot. The term “at least part of”, for example, when used in reference to analyzing an amplification product, means that the entire amplification product can be analyzed, one or both of the individual strands of a double-stranded amplification product can be analyzed, or a fragment, portion, or subsequence of an amplification product can be analyzed.

The term “corresponding” as used herein refers to at least one specific relationship between the elements to which the term relates. For example, a reverse primer of a particular primer pair corresponds to the forward primer of the same primer pair, and vice versa. At least one amplification product primer is designed to anneal with the primer-binding portion of at least one corresponding amplicon. The target-specific portions of the reverse target-specific primers are designed to selectively hybridize with a complementary or substantially complementary region of the corresponding downstream target region flanking sequence. A particular affinity tag binds to the corresponding affinity tag, for example but not limited to, biotin binding to streptavidin. A particular hybridization tag anneals with its corresponding hybridization tag complement; and so forth.

As used herein, the term “degree of methylation” when used in reference to a gDNA target region, refers to the amount of that target region within a sample that is methylated relative to the amount of the same target region that is not methylated, or to the relative number of methylated nucleotides in a target region, or both. In certain embodiments, a sample contains a target region that is fully methylated, a target region that is unmethylated, a target region that has some copies that are fully methylated and some copies that are unmethylated. In some embodiments, a sample comprises copies of a target region that have some but not all of its target nucleotides methylated (intermediate methylation), including some copies with one amount of intermediate methylation and some other copies with at least one different level of intermediate methylation. In some embodiments, determining the degree of methylation for a particular target region comprises obtaining the ratio of methylated target region to unmethylated target region, for example but not limited to, the ratio between the peak height of an amplicon derived from a methylated target region relative to the peak height of an amplicon derived from the same, but unmethylated target region. In certain embodiments, determining the degree of methylation for a particular target region comprises identifying the number or methylated nucleotides in the target region, for example but not limited to evaluating the incremental mobility shift of an amplicon comprising at least one “mobility shifting analog” or “MSA” and calculating the number of incorporated MSAs based on the size of the incremental mobility shift to determine the number of methylated nucleotides in the target region from which the amplicon was derived.

The terms “denaturing” or “denaturation” as used herein refer to any process in which a double-stranded polynucleotide, including a double-stranded amplification product or a double-stranded gDNA fragment is converted to two single-stranded polynucleotides. Denaturing a double-stranded polynucleotide includes without limitation, a variety of thermal and chemical techniques for denaturing a duplex, thereby releasing its two single-stranded components. Those in the art will appreciate that the denaturing technique employed is generally not limiting unless it inhibits or appreciably interferes with a subsequent amplifying and/or determining step.

The term “DNA polymerase” is used in a broad sense herein and refers to any polypeptide that is able to catalyze the addition of deoxyribonucleotides or analogs of deoxyribonucleotides to a nucleic acid polymer in a template dependent manner. For example but not limited to, the sequential addition of deoxyribonucleotides to the 3′-end of a primer that is annealed to a nucleic acid template during a primer extension reaction. Typically DNA polymerases include DNA-dependent DNA polymerases and RNA-dependent DNA polymerases, including reverse transcriptases. Certain reverse transcriptases possess DNA-dependent DNA polymerase activity under certain reaction conditions, including AMV reverse transcriptase and MMLV reverse transcriptase. Such reverse transcriptases with DNA-dependent DNA polymerase activity may be suitable for use with the disclosed methods and are expressly within the contemplation of the current teachings. Descriptions of DNA polymerases can be found in, among other places, Lehninger Principles of Biochemistry, 3d ed., Nelson and Cox, Worth Publishing, New York, N.Y., 2000, particularly Chapters 26 and 29: Twyman, Advanced Molecular Biology: A Concise Reference, Bios Scientific Publishers, New York, N.Y., 1999; Ausubel et al., Current Protocols in Molecular Biology, John Wiley & Sons, Inc., including supplements through May 2005 (hereinafter “Ausubel et al.”); Lin and Jaysena, J. Mol. Biol. 271:100-11, 1997; Pavlov et al., Trends in Biotechnol. 22:253-60, 2004; and Enzymatic Resource Guide: Polymerases, 1998, Promega, Madison, Wis. Expressly within the intended scope of the term DNA polymerase are enzymatically active mutants or variants thereof, including enzymes modified to confer different temperature-sensitive properties (see, e.g., U.S. Pat. Nos. 5,773,258; 5,677,152; and 6,183,998; and DNA Amplification: Current Techniques and Applications, Demidov and Broude, eds., Horizon Bioscience, 2004, particularly in Chapter 1.1).

The term “methylated amplicon” refers to an amplification product that is derived from a target region that comprises at least one methylated target nucleotide, for example but not limited to a 5 mC. A methylated amplicon can be either double-stranded or single-stranded and can be a first amplification product, a second amplification product, or both. The term “unmethylated amplicon” refers to an amplification product that is derived from a target region that does not comprise a methylated target nucleotide. An unmethylated amplicon can be either double-stranded or single-stranded and can be a first amplification product, a second amplification product, or both.

In certain embodiments, a gDNA sample comprising at least one target region is treated with a modifying agent to obtain a modified sample comprising at least one modified target nucleotide. The term “modifying agent” refers to any reagent that can modify a nucleic acid, for example but not limited to at least one target nucleotide in at least one gDNA target region. Some modifying agents convert an unmethylated target nucleotide to a modified nucleotide, but do not convert a methylated target nucleotide to a modified nucleotide (at least not to a significant degree).

In certain embodiments, bisulfite is employed as a modifying agent. Incubating nucleic acid sequences such as gDNA with bisulfite results in deamination of a substantial portion of unmethylated cytosines, which converts such cytosines to uracil. Methylated cytosines are deaminated to a measurably lesser extent. In certain embodiments, the sample is then amplified, resulting in the uracil bases being replaced with thymine. Thus, in certain embodiments, a substantial portion of unmethylated target cytosines ultimately become thymines, while a substantial portion of methylated cytosines remain cytosines. In certain embodiments, the presence of a modified nucleotide (for example but not limited to, uracil or thymine) in the target region may be determined using the methods described in the present teachings. Descriptions of bisulfite treatment can be found in, among other places, U.S. Pat. Nos. 6,265,171 and 6,331,393; Boyd and Zon, Anal. Biochem. 326: 278-280, 2004; U.S. Provisional Patent Application Ser. Nos. 60/499,113; 60/520,942; 60/499,106; 60/523,054; 60/498,996; 60/520,941; 60/499,082; and 60/523,056.

The term “reporter group” is used in a broad sense herein and refers to any identifiable tag, label, or moiety. The skilled artisan will appreciate that many different species of reporter groups can be used in the present teachings, either individually or in combination with one or more different reporter group.

In this application, a statement that one sequence is the same as, substantially the same as, complementary to, or substantially complementary to another sequence encompasses situations where both of the sequences are completely the same as, substantially the same as, or complementary or substantially complementary to one other, and situations where only a portion of one of the sequences is the same as, substantially the same as, complementary to, or substantially complementary to a portion or the entire other sequence. For the purposes of this definition, the term “sequence” includes nucleic acid sequences, polynucleotides, oligonucleotides, primer, target-specific portions, amplification product-specific portions, primer-binding sites, hybridization tags. And hybridization tag complements.

### Certain Exemplary Components

The term “sample” is used in a broad sense herein and is intended to include a wide range of biological materials as well as compositions derived or extracted from such biological materials comprising or suspected of comprising gDNA. Exemplary samples include whole blood; red blood cells; white blood cells; buffy coat; hair; nails and cuticle material; swabs, including buccal swabs, throat swabs, vaginal swabs, urethral swabs, cervical swabs, rectal swabs, lesion swabs, abscess swabs, nasopharyngeal swabs, and the like; urine; sputum; saliva; semen; lymphatic fluid; amniotic fluid; cerebrospinal fluid; peritoneal effusions; pleural effusions; fluid from cysts; synovial fluid; vitreous humor; aqueous humor; bursa fluid; eye washes; eye aspirates; plasma; pulmonary lavages; lung aspirates; and tissues, including, liver, spleen, kidney, lung, intestine, brain, heart, muscle, pancreas, biopsy material, and the like. The skilled artisan will appreciate that lysates, extracts, or material obtained from any of the above exemplary biological samples are also within the scope of the current teachings. Tissue culture cells, including explanted material, primary cells, secondary cell lines, and the like, as well as lysates, extracts, or materials obtained from any cells, are also within the meaning of the term biological sample as used herein. Materials comprising or suspected of comprising at least one gDNA target region that are obtained from forensic, agricultural, and/or environmental settings are also within the intended meaning of the term sample. In certain embodiments, a sample comprises a synthetic nucleic acid sequence. In some embodiments, a sample is totally synthetic, for example but not limited to a control sample comprising a buffer solution containing at least one synthetic nucleic acid sequence.

The first amplification compositions of the current teachings comprise gDNA that includes at least one target region located between a corresponding first flanking sequence and a second flanking sequence. The “first target flanking sequence” is typically located upstream from, i.e., on the 5′ side of, the target region and the corresponding “second target flanking sequence” is typically located downstream from, i.e., on the 3′ side of, the target region. For illustration purposes, the orientation of an illustrative target region relative to its two target flanking sequences is: 5′-first target flanking sequence-target region-second target flanking sequence-3′. It is to be understood that the target flanking sequences can, but need not, be contiguous with the target region. Thus, additional nucleotides may be present between a target flanking sequence and the target region. The target-binding portion of the forward target-specific primer comprises a sequence that is designed to selectively hybridize with the complement of the first target flanking sequence or a sub-sequence within the first target flanking sequence. The target-binding portion of the reverse target-specific primer comprises a sequence that is designed to selectively hybridize with the second target flanking sequence or a sub-sequence within the second target flanking sequence.

The term “target region” refers to the gDNA segment that is being amplified and analyzed to determine the presence or absence of methylated nucleotides and infer or predict the degree of target region methylation. A target region may be located in the promoter or regulatory elements of a gene of interest that is known or suspected of being methylated under certain physiological conditions. The target region is generally located between two flanking sequences, a first target flanking region and a second target flanking region, located on either side of, but not necessarily immediately adjacent to, the target region. In some embodiments, a gDNA segment comprises a plurality of different target regions. In some embodiments, a target region is contiguous with or adjacent to one or more different target regions. In some embodiments, a given target region can overlap a first target region on its 5′-end, a second target region on its 3′-end, or both.

A target region can be either synthetic or naturally occurring. Certain target regions, including flanking sequences where appropriate, can be synthesized using oligonucleotide synthesis methods that are well-known in the art. Detailed descriptions of such techniques can be found in, among other places, Current Protocols in Nucleic Acid Chemistry, Beaucage et al., eds., John Wiley & Sons, New York, N.Y., including updates through May 2005 (hereinafter “Beaucage et al.”); and Blackburn and Gait. Automated DNA synthesizers useful for synthesizing target regions and primers are commercially available from numerous sources, including for example, the Applied Biosystems DNA Synthesizer Models 381A, 391, 392, and 394 (Applied Biosystems, Foster City, Calif.). Target regions, including flanking regions where appropriate, can also be generated biosynthetically, using in vivo methodologies and/or in vitro methodologies that are well known in the art. Descriptions of such technologies can be found in, among other places, Sambrook et al., Molecular Cloning, A Laboratory Manual, Cold Spring Harbor Press (1989) (hereinafter “Sambrook et al.”); and Ausubel et al. Genomic DNA can also be obtained from biological materials using any sample preparation technique known in the art. Purified or partially purified gDNA is commercially available from numerous sources, including Coriell Cell Repositories, Coriell Institute for Medical Research, Camden, N.J.; Serologicals Corp., Norcross, Ga.; and the American Type Culture Collection (ATCC), Manassas, Va.

As used herein, the terms “polynucleotide”, “oligonucleotide”, and “nucleic acid” are used interchangeably and refer to single-stranded and double-stranded polymers of nucleotide monomers, including 2′-deoxyribonucleotides (DNA) and ribonucleotides (RNA) linked by internucleotide phosphodiester bond linkages, or internucleotide analogs, and associated counter ions, e.g., H+, NH4+, trialkylammonium, Mg2+, Na+, and the like. A polynucleotide may be composed entirely of deoxyribonucleotides, entirely of ribonucleotides, or chimeric mixtures thereof. The nucleotide monomer units may comprise any of the nucleotides described herein, including, but not limited to, nucleotides and nucleotide analogs. Polynucleotides typically range in size from a few monomeric units, e.g. 5-40 when they are sometimes referred to in the art as oligonucleotides, to several thousands of monomeric nucleotide units. Unless denoted otherwise, whenever a polynucleotide sequence is represented, it will be understood that the nucleotides are in 5′ to 3′ order from left to right and that “A” denotes deoxyadenosine, “C” denotes deoxycytosine or possibly 5-methyldeoxycytosine (5mC), “G” denotes deoxyguanosine, “T” denotes thymidine, and “U” denotes deoxyuridine, unless otherwise noted.

The term “nucleotide base”, as used herein, refers to a substituted or unsubstituted aromatic ring or rings. In certain embodiments, the aromatic ring or rings contain a nitrogen atom. In certain embodiments, the nucleotide base is capable of forming Watson-Crick or Hoogsteen-type hydrogen bonds with a complementary nucleotide base. Exemplary nucleotide bases and analogs thereof include naturally-occurring nucleotide bases adenine, guanine, cytosine, 5mC, uracil, and thymine, and analogs of the naturally occurring nucleotide bases, including, 7-deazaadenine, 7-deazaguanine, 7-deaza-8-azaguanine, 7-deaza-8-azaadenine, N6-Δ2-isopentenyladenine (6iA), N6-Δ2-isopentenyl-2-methylthioadenine (2ms6iA), N2-dimethylguanine (dmG), 7-methylguanine (7mG), inosine, nebularine, 2-aminopurine, 2-amino-6-chloropurine, 2,6-diaminopurine, hypoxanthine, pseudouridine, pseudocytosine, pseudoisocytosine, 5-propynylcytosine, isocytosine, isoguanine, 7-deazaguanine, 2-thiopyrimidine, 6-thioguanine, 4-thiothymine, 4-thiouracil, O6-methylguanine, N6-methyladenine, O4-methylthymine, 5,6-dihydrothymine, 5,6-dihydrouracil, pyrazolo[3,4-D]pyrimidines (see, e.g., U.S. Pat. Nos. 6,143,877 and 6,127,121 and PCT Published Application WO 01/38584), ethenoadenine, indoles such as nitroindole and 4-methylindole, and pyrroles such as nitropyrrole. Non-limiting examples of nucleotide bases can be found, e.g., in Fasman, 1989, Practical Handbook of Biochemistry and Molecular Biology, pp. 385-394, CRC Press, Boca Raton, Fla., and the references cited therein.

The term “nucleotide”, as used herein, refers to a compound comprising a nucleotide base linked to the C-1′ carbon of a sugar, such as ribose, arabinose, xylose, and pyranose, and sugar analogs thereof. The term nucleotide also encompasses nucleotide analogs. The sugar may be substituted or unsubstituted. Substituted ribose sugars include, but are not limited to, those riboses in which one or more of the carbon atoms, for example the 2′-carbon atom, is substituted with one or more of the same or different, —R, —OR, —NR2 azide, cyanide or halogen groups, where each R is independently H, C1 C6 alkyl, C2 C7 acyl, or C5 C14 aryl. Exemplary riboses include, but are not limited to, 2′-(C1-C6)alkoxyribose, 2′-(C5-C14)aryloxyribose, 2′,3′-didehydroribose, 2′-deoxy-3′-haloribose, 2′-deoxy-3′-fluororibose, 2′-deoxy-3′-chlororibose, 2′-deoxy-3′-aminoribose, 2′-deoxy-3′-(C1-C6)alkylribose, 2′-deoxy-3′-(C1-C6)alkoxyribose and 2′-deoxy-3′-(C5-C14)aryloxyribose, ribose, 2′-deoxyribose, 2′,3′-dideoxyribose, 2′-haloribose, 2′-fluororibose, 2′-chlororibose, and 2′-alkylribose, e.g., 2′-O-methyl, 4′-a-anomeric nucleotides, 1′-a-anomeric nucleotides, 2′-4′- and 3′-4′-linked and other “locked” or “LNA”, bicyclic sugar modifications (see, e.g., PCT Published Application Nos. WO 98/22489, WO 98/39352, and WO 99/14226, and Braasch and Corey, Chem. Biol. 8:1-7, 2001). “LNA” or “locked nucleic acid” is a DNA analogue that is conformationally locked such that the ribose ring is constrained by a methylene linkage between, for example but not limited to, the 2′-oxygen and the 3′- or 4′-carbon or a 3′-4′ LNA with a 2′-5′ backbone (see, e.g., U.S. Pat. Nos. 6,268,490 and 6,670,461). The conformation restriction imposed by the linkage often increases binding affinity for complementary sequences and increases the thermal stability of such duplexes. Exemplary LNA sugar analogs within a polynucleotide include the structures:

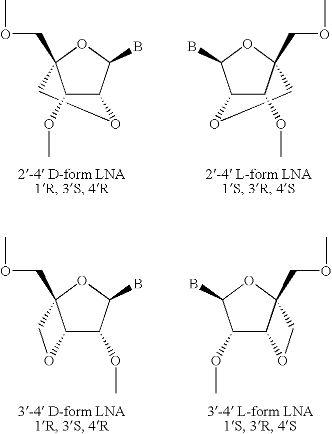

where B is any nucleotide base.

The term “mobility shifting analog” or “MSA” refers to a nucleotide analog of dATP, dCTP, dGTP, or dTTP, that when incorporated into an amplicon detectably changes the migration rate or the amplicon in an analyzing technique, such as a mobility dependent analysis technique, relative to an amplicon comprising the same sequence but with the natural nucleotides not the MSA(s). In other words, the amplicon comprising the incorporated MSA migrates at a different position in at least one analysis technique than would be expected from its length. In some embodiments, an amplicon comprising a MSA migrates faster than its counterpart lacking the MSA. In other embodiments, an amplicon comprising an MSA migrates more slowly that its counterpart lacking the MSA. Non-limiting examples of nucleotide analogs that may be suitable for inducing a mobility shift include boranotriphosphates (including α-P-boranotriphosphates), thiotriphosphates (including deoxy-5′-(α-thio)tri phosphate, e.g., dCTPαS), nucleotide analogs comprising long linker arms, for example but not limited to, (CH2)n and/or (OCH2CH2)n, including biotin-11-dCTP, biotin-11-dCTP, biotin-11-dUTP, digoxigenin-11-dUTP, biotin-aminohexylacrylamido-dCTP (biotin-aha-dCTP), biotin-aha-dUTP, biotin-14-dCTP, biotin-36-dUTP, biotin-36-dCTP, biotin-36-dcATP, and heterocycles, for example but not limited to biotin, N-substituted biotin, and homobiotin cognates, heterocyclic derivatives of hydrocarbon, and polyethylene glycol cognates. Those in the art will appreciate that the suitability of a particular nucleotide analog for use as a MSA depends at in part on the target region, the DNA polymerase used for the amplification reaction, the mobility shift imparted by each analog, the separation and/or detection means, the software. or combinations thereof. Those in the art will understand that the suitability of one or more MSAs can be empirically evaluated, for example using target regions of known methylation state as the starting materials and performing one or more of the disclosed methods under the desired or various reaction conditions, without undue experimentation.

The term “primer” refers to a polynucleotide that selectively hybridizes to a gDNA target flanking sequence or to a corresponding primer-binding site of an amplification product; and allows the synthesis of a sequence complementary to the corresponding polynucleotide template from its 3′ end.

A “target-specific primer pair” of the current teachings comprises a forward target-specific primer and a reverse target-specific primer. The forward target-specific primer comprises a first target-specific portion that comprises a sequence that is the same as or substantially the same as the nucleotide sequence of the first or upstream target flanking sequence, and that is designed to selectively hybridize with the complement of the upstream target flanking sequence that is present in, among other places, the reverse strand amplification product. In some embodiments, the forward target-specific primer further comprises a first tail portion, located upstream from the first target-specific portion, that comprises a first primer-binding site. The reverse target-specific primer of the primer pair comprises a second target region-specific portion that comprises a sequence that is complementary to or substantially complementary to, and that is designed to selectively hybridize with, the second or downstream target region flanking sequence. In some embodiments, the reverse target-specific primer further comprises a second tail portion, located upstream from the second target-specific portion, that comprises a second primer-binding site. In some embodiments, the tail portion of a reverse target-specific primer further comprises a sequence that is designed to enhance the non-templated addition of nucleotides, typically A, to the end of a primer extension product by certain DNA polymerases, sometimes referred to as the Clark reaction (see, e.g., Clark, Nucl. Acids Res. 16(20):9677-84, 1988). Some non-limiting examples of such sequences include GTTTCTT, GTTT, and GTT, sometimes referred to as PIGtail sequences (see, e.g., Brownstein et al., BioTechniques 2)(6):1004-10, 1996), or a single G at the 5′-end of a tailed primer. In certain embodiments, at least one forward target-specific primer, at least one reverse target-specific primer, or at least one forward target-specific primer and at least one reverse target-specific primer further comprises at least one of: a reporter probe-binding site, an additional primer-binding site, and a reporter group, for example but not limited to a fluorescent reporter group. In certain embodiments, a forward primer and the corresponding reverse primer of a target-specific primer pair have different melting temperatures (Tm) to permit temperature-based asymmetric PCR.

In some embodiments, a target-specific primer pair comprises (1) a forward target-specific primer comprising a first target-binding portion that is the same as or substantially the same as a first target flanking sequence, located upstream (5′) of the gDNA target region and (2) a corresponding reverse target-specific primer comprising a second target-binding portion that is complementary to or substantially complementary to a corresponding second target flanking sequence, located downstream (3′) of the same gDNA target region. In some embodiments, a target-specific primer pair, includes (1) a forward target-specific primer comprising (a) a first target-binding portion that is the same as or substantially the same as a first target flanking sequence, located upstream (5′) of the gDNA target region and (b) a first tail portion located upstream from the first target-binding portion, wherein the tail sequence comprises a first primer-binding site; and (2) a corresponding reverse target-specific primer comprising (a) a second target-binding portion that is complementary to or substantially complementary to a corresponding second target flanking sequence, located downstream (3′) of the same gDNA target region and (b) a second tail sequence located upstream from the second target-binding sequence, wherein the second tail sequence comprises a second primer-binding site.

Those in the art will appreciate that treatment of gDNA with certain modifying agents, for example but not limited to sodium bisulfite, cause unmethylated C to be deaminated to U. A gDNA flanking region comprising an unmethylated C would, after sodium bisulfite treatment, result in a modified nucleotide in the flanking region of modified sample, which could prevent or decrease the ability of the corresponding target-specific primer to selectively hybridize. The target-specific primers of the current teachings are typically designed to selectively hybridize with target flanking sequences that are outside CpG islands to allow a target region amplicon to be generated regardless of the methylation state of the target region.

The term “amplification product primer pair” refers to a forward amplification product primer and a corresponding reverse amplification product primer. In some embodiments, an amplification primer pair comprises a universal primer or a universal primer pair and the same primer pair is used to amplify at least two different species of amplification product. In some embodiments, an amplification product primer pair comprises a forward primer and a reverse primer that are designed to amplify one amplification product species. For example but without limitation, a first amplification product primer pair comprising a forward first amplification product primer comprising a sequence that is designed to selectively hybridize with the complement of an upstream primer-binding site of a particular single-stranded first amplification product species and a reverse first amplification product primer that is designed to selectively hybridize with the corresponding downstream primer-binding site of the same single-stranded first amplification product species. In some embodiments, an amplification product primer pair is designed to selectively hybridize with corresponding regions of an amplification product or its complement that are internal to the binding sites of the target-specific primer pair, including a nested primer pair, or to binding sites that partially overlap the binding sites of the target-specific primer pair. In certain embodiments, at least one forward amplification product primer, at least one reverse amplification product primer, or at least one forward amplification product primer and at least one reverse amplification product primer further comprises at least one of: a reporter probe-binding site, an additional primer-binding site, and a reporter group, for example but not limited to a fluorescent reporter group. In certain embodiments, a forward primer and the corresponding reverse primer of an amplification product primer pair have different melting temperatures to permit temperature-based asymmetric PCR.

In certain embodiments, one or more of a primer's components may overlap or partially overlap one or more other primer components. For example but not limited to, a target-specific portion may overlap or partially overlap a primer-binding site, a reporter probe-binding site, a hybridization tag, an affinity tag, a reporter group.

The skilled artisan will appreciate that the complement of the disclosed gDNA target regions, primers, target-specific portions, primer-binding sites, or combinations thereof, may be employed in certain embodiments of the present teachings. For example, without limitation, a particular gDNA may comprise both the gDNA target region and its complement. Thus, in certain embodiments, when a gDNA sample is denatured, both the target region and its complement are present in the sample as single-stranded sequences and either or both of the single-stranded sequences can be amplified and analyzed. Those in the art will appreciate, however, that in certain circumstances, a double-stranded gDNA segment comprising a target region may be hemimethylated. For example, but not as a limitation, one strand of the double-stranded gDNA segment may comprise a methylated target nucleotide while the corresponding target nucleotide in the complementary gDNA strand is unmethylated. In certain embodiments, it is desirable to determine the degree of methylation of both the target region and its complement to obtain an accurate understanding of the methylation state of the gDNA segment in question.

As used herein, the terms “forward” and “reverse” are used to indicate relative orientation of the corresponding primers of a primer pair on a polynucleotide sequence. For illustration purposes but not as a limitation, consider a single-stranded polynucleotide drawn in a horizontal, left to right orientation with its 5′-end on the left. The “reverse” primer is designed to anneal with the downstream primer-binding site at or near the “3′-end” of this illustrative polynucleotide, in a 5′ to 3′ orientation, right to left. The corresponding “forward primer is designed to anneal with the complement of the upstream primer-binding site at or near the “5′-end” of the polynucleotide, in a 5′ to 3′ “forward” orientation, left to right. Thus, the reverse primer comprises a sequence that is complementary to the reverse or downstream primer-binding site of the polynucleotide and the forward primer comprises a sequence that is the same as the forward or upstream primer-binding site. It is to be understood that the terms “3′-end” and “5′-end”, as used in this paragraph, are illustrative only and do not necessarily refer literally to the respective ends of the polynucleotide. Rather, the only limitation is that the reverse primer of this exemplary primer pair anneals with a reverse primer-binding site that is downstream or to the right of the forward primer-binding site that comprises the same sequence or substantially the same sequence as the corresponding forward primer. As will be recognized by those of skill in the art, these terms are not intended to be limiting, but rather to provide illustrative orientation in a given embodiment.

As used herein, the term “primer-binding site” refers to a region of a polynucleotide sequence such as a tailed primer or an amplification product that can serve directly, or by virtue of its complement, as the template upon which a primer can anneal for any of a variety of primer extension reactions known in the art, for example but not limited to, PCR. When a tailed primer comprises a primer-binding site, it is typically located upstream from a sequence-specific binding portion of the primer, for example but not limited to, the first target-binding portion of a forward target-specific primer or the second primer-binding portion of a reverse amplification product primer.

Those in the art appreciate that as an amplification product is amplified by certain amplification techniques, the complement of the primer-binding site is synthesized in the complementary strand. Thus, it is to be understood that the complement of a primer-binding site is expressly included within the intended meaning of the term primer-binding site, unless stated otherwise.

In some embodiments, a multiplicity of different primer pairs are employed in an amplifying step, for example but not limited to a multiplex amplification reaction, wherein the different primer pairs are designed to amplify a multiplicity of different nucleotide sequences, including a multiplicity of different gDNA target regions or a multiplicity of different amplification products.

The skilled artisan will appreciate that while the primers and primer pairs of the present teachings may be described in the singular form, a plurality of primers may be encompassed by the singular term. Thus, for example, in certain embodiments, a target-specific primer pair typically comprises a plurality of forward target-specific primers and a plurality of corresponding reverse target-specific primers.

In some embodiments, a primer and/or an amplification product comprise an affinity tag. In some embodiments, an affinity tag comprises a reporter group. In certain embodiments, affinity tags are used for separating, are part of a detecting means, or both.

In some embodiments, at least one of: a primer, a MSA, and an amplification product comprise a mobility modifier. In certain embodiments, mobility modifiers comprise nucleotides of different lengths effecting different mobilities. In certain embodiments, mobility modifiers comprise non-nucleotide polymers, for example but not limited to, polyethylene oxide (PEO), polyglycolic acid, polyurethane polymers, polypeptides, and oligosaccharides. In certain embodiments, mobility modifiers may work by adding size to a polynucleotide, or by increasing the “drag” of the molecule during migration through a medium without substantially adding to the size. Certain mobility modifiers, including PEO's, have been described in, among other places, U.S. Pat. Nos. 5,470,705; 5,580,732; 5,624,800; and 5,989,871 and United States Patent Application Publication No. US 2003/0190646 Al.

### Certain Exemplary Component Techniques

According to the instant teachings, gDNA may be obtained from any living, or once living, organism, including a prokaryote, an archaea, or a eukaryote, for example but not limited to, an insect including Drosophila, a worm including C. elegans, a plant, and an animal, including a human; and including prokaryotic cells and cells, tissues, and organs obtained from a eukaryote, for example but not limited to, cultured cells and blood cells. Certain viral genomic DNA is also within the scope of the current teachings. In certain embodiments, the gDNA may be present in a double-stranded or single-stranded form. The skilled artisan appreciates that gDNA includes not only full length material, but also fragments generated by any number of means, for example but not limited to, enzyme digestion, sonication, shear force, and the like, and that all such material, whether full length or fragmented, represent forms of gDNA that can serve as templates for an amplifying reaction of the current teachings.

A variety of methods are available for obtaining gDNA for use with the current teachings. Methylated and unmethylated gDNA is also commercially available. When the gDNA is obtained through isolation from a biological matrix, preferred isolation techniques include (1) organic extraction followed by ethanol precipitation, e.g., using a phenol/chloroform organic reagent (see, e.g., Sambrook et al.; Ausubel et al.), for example using an automated DNA extractor, e.g., the Model 341 DNA Extractor (Applied Biosystems, Foster City, Calif.); (2) stationary phase adsorption methods (e.g., Boom et al., U.S. Pat. No. 5,234,809; Walsh et al., Biotechniques 10(4): 506-513, 1991); and (3) salt-induced DNA precipitation methods (see, e.g., Miller et al., Nucl. Acids Res. 16(3): 9-10, 1988), such precipitation methods being typically referred to as “salting-out” methods. In certain embodiments, gDNA isolation techniques comprise an enzyme digestion step to help eliminate unwanted protein from the sample, for example but not limited to, digestion with proteinase K, or other like proteases; a detergent; or both (see, e.g., U.S. Patent Application Publication 2002/0177139; and U.S. patent application Ser. Nos. 09/724,613 and 10/618,493). Commercially available nucleic acid extraction systems include, among others, the ABI PRISM® 6700 Nucleic Acid PrepStation and the ABI PRISM® 6700 Nucleic Acid Automated Work Station; nucleic acid sample preparation reagents and kits are also commercially available, including, NucPrep™ Chemistry, BloodPrep™ Chemistry, the ABI PRISM® TransPrep System, and PrepMan™ Ultra Sample Preparation Reagent (all from Applied Biosystems).

The term “mobility-dependent analysis technique” refers to any analysis method based on different rates of migration between different analytes. Non-limiting examples of mobility-dependent analysis techniques include chromatography, sedimentation, gradient centrifugation, field-flow fractionation, multi-stage extraction techniques, mass spectrometry, and electrophoresis, including slab gel, isoelectric focusing, and capillary electrophoresis.

The terms “amplifying” and “amplification” are used in a broad sense and refer to any technique by which a target region, an amplicon, or at least part of an amplicon, is reproduced or copied (including the synthesis of a complementary strand), typically in a template-dependent manner, including a broad range of techniques for amplifying nucleic acid sequences, either linearly or exponentially. Some non-limiting examples of amplification techniques include primer extension, including the polymerase chain reaction (PCR), RT-PCR, asynchronous PCR (A-PCR), and asymmetric PCR, strand displacement amplification (SDA), multiple displacement amplification (MDA), nucleic acid strand-based amplification (NASBA), rolling circle amplification (RCA), transcription-mediated amplification (TMA), and the like, including multiplex versions and/or combinations thereof. Descriptions of certain amplification techniques can be found in, among other places, Molecular Cloning, A Laboratory Manual, Cold Spring Harbor Press, 3d ed., 2001 (hereinafter “Sambrook and Russell”); Sambrook et al.; Ausubel et al.; PCR Primer: A Laboratory Manual, Diffenbach, Ed., Cold Spring Harbor Press (1995); Msuih et al., J. Clin. Micro. 34:501-07 (1996); McPherson; Rapley; U.S. Pat. Nos. 6,027,998 and 6,511,810; PCT Publication Nos. WO 97/31256 and WO 01/92579; Ehrlich et al., Science 252:1643-50 (1991); Favis et al., Nature Biotechnology 18:561-64 (2000); Protocols & Applications Guide, rev. 9/04, Promega, Madison, Wis.; and Rabenau et al., Infection 28:97-102 (2000).

The terms “amplification product” and “amplicon” are essentially used interchangeably herein and refer to the nucleic acid sequences generated from any cycle of amplification of any amplification reaction, for example a first amplicon is generated during a first amplification reaction and a second amplicon product is generated during a second amplification reaction, unless otherwise apparent from the context. An amplicon can be either double-stranded or single-stranded, including the separated component strands obtained from a double-stranded amplification product.

In certain embodiments, amplification techniques comprise at least one cycle of amplification, for example, but not limited to, the steps of: selectively hybridizing a primer to a target region flanking sequence or a primer-binding site of an amplicon (or complements of either, as appropriate); synthesizing a strand of nucleotides in a template-dependent manner using a polymerase; and denaturing the resulting nucleic acid duplex to separate the strands. The cycle may or may not be repeated.

Amplification can comprise thermocycling or can be performed isothermally. In some embodiments, amplifying comprises a thermocycler, for example but not limited to a GeneAmp® PCR System 9700, 9600, 2700, or 2400 thermocycler (all from Applied Biosystems). In some embodiments, double-stranded amplification products are not initially denatured, but are used in their double-stranded form in one or more subsequent steps. In certain embodiments, single-stranded amplicons are generated in an amplification reaction, for example but not limited to asymmetric PCR or A-PCR.

The term “analyzing” when used in reference to a first amplicon, part of a first amplicon, a second amplicon, part of a second amplicon, or combinations thereof, includes any technique that allows one or more parameter of an amplicon or at least part of an amplicon to be obtained. In certain embodiments, analyzing comprises (1) separating (at least partially) one amplicon species from another amplicon species, including amplicons derived from different target regions and amplicons derived from the same target region but with different degrees of methylation (e.g., fully methylated, unmethylated, and intermediate levels of methylation, sometimes referred to as a group or family of “related amplicons”), (2) detecting a separated and/or partially separated amplicon, and (3) obtaining and evaluating one or more amplicon parameter, for example but not limited to, amplicon peak height, integrated area under an amplicon peak, and amplicon intensity, including the fluorescent intensity of an incorporated fluorescent reporter group, the luminescent intensity of an incorporated bioluminescent, chemiluminescent and/or phosphorescent reporter group, and the radioactive intensity of an incorporated isotope. Typically, one or more parameter(s) of one amplicon is compared with the same parameter(s) of another amplicon to determine the degree of target region methylation, including qualitative, semi-quantitative, and quantitative determinations. The degree of methylation of at least one target region is typically determined by inference, for example but not limited to, by determining whether an amplicon derived from a modified sample comprises a modified nucleotide or its complement and inferring that the corresponding target region is methylated or is not methylated.

### Certain Exemplary Embodiments

Reference will now be made to various exemplary embodiments, examples of which are illustrated in the accompanying drawings. Wherever possible, the same reference numbers are used in the drawings and the description to refer to the same or like parts.

The present teachings enable analysis of data subsequent to a novel method of methylation detection. The present teachings provide methylation prediction systems and methods. More specifically, the present teaching provide systems and methods for predicting mobility differences between amplicons of methylated and unmethylated gDNA. Even further, the present teachings provide systems and methods for predicting size of an amplicon for both (treated and untreated) fragments.

The method for predicting a methylation of a target can be by determining the degree of methylation of at least one target region and for quantitating the number of methylated nucleotides in a given target region, by modifying certain target nucleotides within the target region and then analyzing the amplicon of that modified target region.

The analytical methodology to detect the presence of methylated CpG in genomic DNA has been developed and is described in the following to the extent needed to explain the present teachings. The methodology is enhanced by algorithmic determinations to predict a degree of methylation of a target region and to predict a size of a methylated or unmethylated component in a target region.

Briefly, fluorescent products arising from separate PCR-amplification reactions of bisulfite treated and untreated gDNA are combined into a single tube and the pooled sample subjected to capillary electrophoresis (CE) in the present of a DNA size standard. Alternatively, a single, bisulfite treated sample containing mixed methylation states (methylated and unmethylated) will co-amplify both amplicons in a PCR amplification. The two PCR products will electrophoretically separate and an observed size is determined for each of the products.

More specifically, and fundamental to understanding the role of cytosine (C) in genomic DNA (gDNA) is the need for robust analysis methods to determine the location and degree of its modification. The present invention utilizes information obtainable by these methods to define a methylation prediction algorithm. With the algorithm, it becomes possible to predict the degree of methylation based on fragment mobility and predict size of methylated fragment components.

In the above-identified corresponding provisional patent applications, and as expanded upon herein, a method for methylation detection by denaturing capillary electrophoresis (CE) is described in using standard fragment analysis conditions. Bisulfite treatment of gDNA will selectively deaminate C, but not 5-methylcytosine (5mC). Amplicons generated form bisulfite-converted gDNA are analyzed immediately after PCR using a 6-carboxy fluorescein (6-FAM) dye-labeled primer. The amplicons from methylated and unmethylated gDNA separate based solely on base composition due to the presence of multiple C vs. T differences. By direct detection of PCR amplicons following PCR using primers that anneal independently of methylation status, the overall workflow from gDNA sample input to data analysis is relatively simple. Further, the same PCR product is suitable for additional analyses such as direct sequencing, cloning and sequencing, single-base extension or post-PCR incorporation of a modified dCTP, the latter of which allows resolution of amplicons with as little as a single C/T difference. An exemplary utility of this novel CE detection assay is shown by analyzing the hypermethylated region of the fragile-X FMR 1 locus.

Further, methylation of the cytosine ring to form 5-methyl cytosine (5mC) in normally unmethylated CpG islands in the promoter region of genes has been associated with transcriptional silencing, and plays a central role in epigenetics as described in P. A. Jones, and D. Takai, The role of DNA methylation in mammalian epigenetics, Science 293 (2001) 1068-1070; J. P. Issa, Methylation and prognosis: of molecular clocks and hypermethylator phenotypes, Clin Cancer Res 9 (2003) 2879-2881; K. L. Novik, I. Nimmrich, B. Genc, S. Maier, C. Piepenbrock, A. Olek, and S. Beck, Epigenomics: genome-wide study of methylation phenomena, Curr Issues Mol Biol 4 (2002) 111-128; G. A. Garinis, G. P. Patrinos, N. E. Spanakis, and P. G. Menounos, DNA hypermethylation: when tumour suppressor genes go silent, Hum Genet. 111 (2002) 115-127; M. Widschwendter, and P. A. Jones, DNA methylation and breast carcinogenesis, Oncogene 21 (2002) 5462-5482; and M. Widschwendter, and P. A. Jones, The potential prognostic, predictive, and therapeutic values of DNA methylation in cancer. Commentary re: J. Kwong et al., Promoter hypermethylation of multiple genes in nasopharyngeal carcinoma. Clin. Cancer Res., 8: 131-137, 2002, and H-Z. Zou et al., Detection of aberrant p16 methylation in the serum of colorectal cancer patients. Clin. Cancer Res., 8: 188-191, 2002, Clin Cancer Res 8 (2002) 17-21.

The importance of gDNA methylation is evident by the increasing number of research publications or reviews and grants awarded each year that deal with various aspects of DNA methylation. Under appropriate conditions, bisulfite treatment of gDNA converts cytosines to uracil (U), without significant conversion of 5 mC as described in P. M. Warnecke, C. Stirzaker, J. Song, C. Grunau, J. R. Melki, and S. J. Clark, Identification and resolution of artifacts in bisulfite sequencing, Methods 27 (2002) 101-107; and C. Grunau, S. J. Clark, and A. Rosenthal, Bisulfite genomic sequencing: systematic investigation of critical experimental parameters, Nucleic Acids Res 29 (2001) E65-65.

PCR amplification of the bisulfite-treated gDNA therefore amplifies 5mC as C and U is “read” as T. Methylation at CpG-rich motifs often affects an entire region and is bimodal, i.e., all or most of the CpGs are either methylated or unmethylated as described in V. K. Rakyan, T. Hildmann, K. L. Novik, J. Lewin, J. Tost, A. V. Cox, T. D. Andrews, K. L. Howe, T. Otto, A. Olek, J. Fischer, I. G. Gut, K. Berlin, and S. Beck, DNA methylation profiling of the human major histocompatibility complex: a pilot study for the human epigenome project PLoS Biol 2 (2004) e405.

Many methylation assays are based on hybridization using primers or probes designed for either the fully methylated or unmethylated state of interest as described in M. Zeschnigk, S. Bohringer, E. A. Price, Z. Onadim, L. Masshofer, and D. R. Lohmann, A novel real-time PCR assay for quantitative analysis of methylated alleles (QAMA): analysis of the retinoblastoma locus, Nucleic Acids Res 32 (2004) e125; C. A. Eads, K. D. Danenberg, K. Kawakami, L. B. Saltz, C. Blake, D. Shibata, P. V. Danenberg, and P. W. Laird, MethyLight: a high-throughput assay to measure DNA methylation, Nucleic Acids Res 28 (2000) E32; and J. G. Herman, J. R. Graff, S. Myohanen, B. D. Nelkin, and S. B. Baylin, Methylation-specific PCR: a novel PCR assay for methylation status of CpG islands, Proc Nat Acad Sci USA 93 (1996) 9821-9826. A disadvantage of hybridization based methods using these primers and probes is the lack of PCR product or signal in cases where methylation is intermediate and variable. Design of primers or probes with mixed bases at possible methylation sites have different annealing temperatures; consequently permissive annealing temperatures encourage mismatches and higher annealing temperatures result in no or biased product(s). Primers designed to anneal to CpG-less sequences outside of CpG motifs of interest will amplify regardless of the methylation states of the bisulfite-converted gDNA. The amplicons can be cloned and sequenced to determine the variable methylation patterns present in the sample. However, intended quantification methods for clinical samples are subject to variability in sample homogeneity, variability in the bisulfite conversion efficiency and isolation of converted DNA, PCR bias as described in P. M. Warnecke, C. Stirzaker, J. R. Melki, D. S. Millar, C. L. Paul, and S. J. Clark, Detection and measurement of PCR bias in quantitative methylation analysis of bisulphite-treated DNA, Nucleic Acids Res 25 (1997) 4422-4426; and K. O. Voss, K. P. Roos, R. L. Nonay, and N. J. Dovichi, Combating PCR bias in bisulfite-based cytosine methylation analysis. Betaine-modified cytosine deamination PCR, Anal Chem 70 (1998) 3818-3823. In the case of cloning and sequencing, cloning bias has been implicated, referring also to A. Meissner, A. Gnirke, G. W. Bell, B. Ramsahoye, E. S. Lander, and R. Jaenisch, Reduced representation bisulfite sequencing for comparative high-resolution DNA methylation analysis, Nucleic Acids Res 33 (2005) 5868-5877.

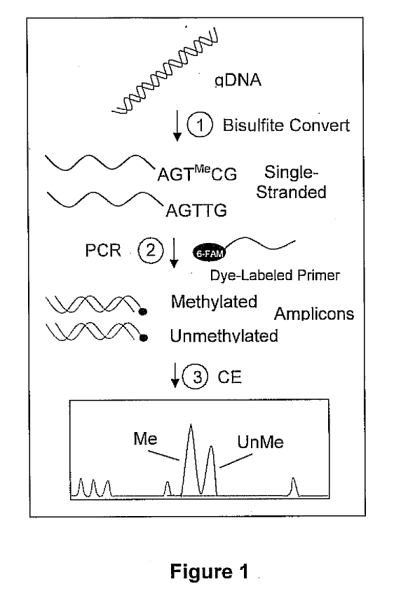

In the following, description is directed to improved PCR amplification efficiency for bisulfite-converted gDNA, reduction of PCR bias of methylated and unmethylated “mixed” samples, and demonstrate that percentage of methylation can be directly observed using capillary electrophoresis (CE). This simple analysis scheme is presented in FIG. 1. Furthermore, the same PCR amplicon is suitable for additional characterization or confirmation such as direct sequencing, cloning and sequencing, or single-base extension.

In general, FIG. 1 is a schematic representation of the workflow for methylation dependent fragment separation (MDFS) having three steps. In step 1, following bisulfite conversion, Me gDNA differs from unmethylated gDNA by the presence of multiple 5mC vs. U bases. In step 2, a region of interest is PCR amplified using a single set of FAM dye-labeled primers that amplify the gDNA regardless of the methylation status. In step 3, the presence of the multiple polymorphisms (C vs. T) leads to differential migration times during fragment analysis by CE so that an amplicon from fully methylated gDNA is readily resolved from an amplicon of fully unmethylated gDNA.

### Example of Bisulfite Conversion and Purification

Bisulfite conversion was performed as previously described in V. L. Boyd, and G. Zon, Bisulfite conversion of genomic DNA for methylation analysis: protocol simplification with higher recovery applicable to limited samples and increased throughput, Anal Biochem 326 (2004) 278-280, but with increased centrifugation times. A maximum of 300 ng of human gDNA (Coriell cell repository http://locus.umdnj.edu) in an initial volume of 45 μL of water was mixed with 5 μL of “M-dilution buffer” (Zymo Research, Orange Calif.) and the solution was heated for 15 min at 37° C. and then kept at 37° C. until ready for use. To the denatured gDNA, 100 μL of “CT conversion reagent” (Zymo, contains sodium bisulfite, an irritant), freshly prepared according to manufacturer's instructions, was added to give a final volume of 150 μL, and the reaction was incubated at 50° C. for 15 h. The entire solution was transferred to a Microcon 100 device (Millipore, Bellerica, Mass.), mixed with ˜150-300 μL of water to reduce the viscosity of the high molarity sodium bisulfite solution, and then centrifuged for 15-20 min at 500×g (2800 rpm in an Eppendorf 5415) until just or nearly dry. Water (350 μL) was added to the upper chamber and centrifugation was resumed until nearly dry. This step was repeated. For desulfonation (in situ) 350 μL of 0.1 M NaOH was added to the upper chamber, and after 5 min at room temperature, the solution was centrifuged until nearly dry. Water (350 μL) was added and centrifugation was continued until near dryness. TE buffer (50 μL of 10 mM Tris-0.1 mM EDTA, pH 8, Teknova) was added to the upper chamber, and the liquid mixed by pipeting up and down several times. After 5-10 min the resultant TE solution of bisulfite-converted gDNA was removed and stored at 4° C. The bisulfite-converted DNA is stable for at least 1 year at 4° C.

### Primer Design

PCR primers for the CpG island regions of 18 genes were selected and the primer sequences are provided in Table 1 shown below. Forward and reverse primers were tailed with the −21M13 forward and reverse sequences. Candidate primers were selected with the aid of MethPrimer as described in L. C. Li, and R. Dahiya, MethPrimer: designing primers for methylation PCRs, Bioinformatics 18 (2002)1427-1431 (http://www.urogene.org/methprimer/). Primer pairs selected by the software that amplified a homopolymer region exceeding 8 sequential Ts were rejected, and replaced with primers that amplified suitable regions upstream or downstream. Primer pair selection was based solely on the criteria that the primers contain no CpG dinucleotides and provide amplicons devoid of regions with poly T≧9. The gene specific portion of the primer typically had a Tₘ of 55±5° C. based on theoretical calculations that were carried out using methodology available at the following website: http-//www.basic.northwestern.edu/biotools/oligocalc.html. The forward primer was fluorescently-labeled (6-FAM™ dye, Applied Biosystems, Foster City, Calif.) for detection during CE.

TABLE 1
Primer sequences (-21M13-tailed) for bisulfite-converted gDNA.¹

| Gene Name | | |
|---|---|---|
| Genebank ID | | |
| (Location) | Primer | M13-tailed primer sequence |
| BRCA | forward | (6-FAM)TGTAAAACGACGGCCAGTATTTGAGAAATTTTATAGTTTGTTTTT |
| U37574 | reverse | GCAGGAAACAGCTATGACCTATTCTAAAAAACTACTACTTAAC |
| (1503-1652) | | |
| SRBC | forward | (6-FAM)TGTAAAACGACGGCCAGTTGGGGTTAATAGGTTTTTTAGTAGG |
| AF408198 | reverse | GCAGGAAACAGCTATGACCAACTCCAACTATAACTCAAACAAAC |
| (3817-3968) | | |
| IMP | forward | (6FAM)GTGTAAAACGACGGCCAGTTGGTTTGGGTTAGAGATATTTAGTG |
| AL023282 | reverse | GCAGGAAACAGCTATGACCTTCAAATCCTTATAAAAAATAATACC |
| (4913-5084) | | |
| CDH1 | forward | (6-FAM)TGTAAAACGACGGCCAGTTTTAGTAATTTTAGGTTAGAGGGTTAT |
| L34545 | reverse | GCAGGAAACAGCTATGACCTAACTACAACCAAATAAACCCC |
| (836-987) | | |
| MYOD1 | forward | (6FAM)GTGTAAAACGACGGCCAGTTTTTGTGTTTTTAATGTTTTGTTTTTTT |
| AF027148 | reverse | GCAGGAAACAGCTATGACCCCTTTCCAAACCTCTCCAACAC |
| (9767-9952) | | |
| RasSF(187) | forward | (6FAM)GTGTAAAACGACGGCCAGTTAGTTTAATGAGTTTAGGTTTTTT |
| AC002481 | reverse | GCAGGAAACAGCTATGACCCTACACCCAAATTTCCATTA |
| (17928-18114)* | | |
| FMR1 | forward | (6FAM)TGTAAAACGACGGCCAGTTGAGTGTATTTTTGTAGAAATGGG |
| X61378 | reverse | GCAGGAAACAGCTATGACCTCTCTCTTCAAATAACCTAAAAAC |
| (2301-2420) | | |
| MGMT | forward | (6FAM)GTGTAAAACGACGGCCAGTATGGTTTTTGGTTTATGAAGGTTAT |
| M29971 | reverse | GCAGGAAACAGCTATGACCAAACACTACCACTTCCTTTAATACAAC |
| (594-822) | | |
| APC | forward | (6-FAM)TGTAAAACGACGGCCAGTATTTTTTTGTTTGTTGGGGATTGGG |
| U02509 | reverse | GCAGGAAACAGCTATGACCAACTACACCAATACAACCACATATC |
| (601-850) | | |
| p16 (CDKN2A) | forward | (6-FAM)TGTAAAACGACGGCCAGTGGTTGGTTGGTTATTAGAG |
| gi 20330501 | reverse | GCAGGAAACAGCTATGACCCCCTCTACCCACCTAAAT |
| (192307-192060) | | |
| ER | forward | (6FAM)GTGTAAAACGACGGCCAGTGTTTTATTGTATTAGATTTAAGGGAA |
| X82462 | reverse | GCAGGAAACAGCTATGACCCTATTAAATAAAAAAAAACCCCCCAAAC |
| (3040-3308) | | |
| MLH1 | forward | (6FAM)GTGTAAAACGACGGCCAGTTTTTTTTAGGAGTGAAGGAGGTTA |
| U26559 | reverse | GCAGGAAACAGCTATGACGCCCAAAAAAAACAAAATAAAAATC |
| (178-451) | | |
| ALX3 | forward | (6-FAM)TGTAAAACGACGGCCAGTTTTAGGTTTTTTTTTTTGG |
| AF008202 | reverse | GCAGGAAACAGCTATGACCCTAAAAAATAAAACTCCAAAAAC |
| (288-562) | | |
| p15 | forward | (6-FAM)TGTAAAACGACGGCCAGTTAGGTTTTTTAGGAAGGAGAG |
| S75756 | reverse | GCAGGAAACAGCTATGACCCTAAAACCCCAACTACCTAAA |
| (340-629) | | |
| COX2 | forward | (6-FAM)TGTAAAACGACGGCCAGTGTTTTTAGATAGTAAAGTTTATTTT |
| D28235 | reverse | GCAGGAAACAGCTATGACCTACTTATAAAAAAACTAAAATATCC |
| (1774-2065) | | |
| DAPk | forward | (6-FAM)TGTAAAACGACGGCCAGTGTTTGTAGGGTTTTTATTGGT |
| gi 15364802 | reverse | GCAGGAAACAGCTATGACCCCCTAACTAAAAAAACAAAAACTAA |
| (46932-47311) | | |
| RB1 | forward | (6-FAM)TGTAAAACGACGGCCAGTTTTTAGTTTAATTTTTTATGATTTAG |
| L11910 | reverse | GCAGGAAACAGCTATGACCTCTAAATCCTCCTCAAAAAAAAA |
| (1750-2160) | | |
| RasSF (451) | forward | (6-FAM)TGTAAAACGACGGCCAGTTTTTGTTTATTTGTGGTTTAGATA |
| AC002481 | reverse | GCAGGAAACAGCTATGACCAAAAAACCTAAACTCATTAAACTA |
| (18022-18541)² | | |

### Polymerase Chain Reactions (PCR)

The primer pair (0.25 μL forward primer, 0.25 μL reverse primer, 5 μM each) was combined with 0.5 μL bisulfite treated gDNA (3 ng/μL, assuming 100% recovery of unfragmented gDNA after the bisulfite conversion), 1 μL AmpliTaq Gold® 10× buffer, 0.8 μL dNTPs (2.5 mM each), 0.8 μL MgCl2 (25 nM), 0.2 μL AmpliTaq Gold® polymerase (5 U/μL, all from Applied Biosystems) and 6.2 μL water. The thermal cycling conditions were 5 min at 95° C. (to activate the hot-start polymerase), 5 cycles of 95° C./30 s, 60° C./2 min, 72° C./3 min; 30 cycles of 95° C./30 s, 65° C./1 min, 72° C./3 min, hold at 60° C./85 min (to allow complete non-templated A addition which is further facilitated by the “C” at the 3′ end as described in J. M. Clark, Novel non-templated nucleotide addition reactions catalyzed by procaryotic and eucaryotic DNA polymerases, Nucleic Acids Res 16 (1988) 9677-9686; and G. Hu, DNA polymerase-catalyzed addition of nontemplated extra nucleotides to the 31 end of a DNA fragment, DNA Cell Biol 12 (1993) 763-770. [18, 19]) and stored at 4° C. The optimum annealing temperature varied slightly for each primer set, but ideally the selected annealing temperature for the initial 5 cycles was chosen to be −5° C. above the calculated Tₘ.

### Capillary Electrophoresis

A 0.5-μL aliquot (or less) of the PCR reaction mixture prepared as described above was added to 12 μL of Hi-Di™ Formamide containing 10% ROX™ 500 size standard (Applied Biosystems), and heated at 95° C. for 5 min to denature the amplicon. Fragments were analyzed at 60° C. on an ABI Prism 3100 GeneAnalyzer using a 36-cm capillary array, POP 4 polymer and GeneMapper® Software for data collection with run module Frag36_POP4_D (all from Applied Biosystems).

### FMR1

For analysis of the methylation pattern of the fragile-X gene (See R. Stoger, T. M. Kajimura, W. T. Brown, and C. D. Laird, Epigenetic variation illustrated by DNA methylation patterns of the fragile-X gene FMR1, Hum Mol Genet 6 (1997) 1791-1801), the following gDNA samples were obtained from the Coriell cell repository (http://locus.umdnj.edu): NA 06852 (fragile-X male), NA 17117 (male, Human Variation Panel), NA 17134 (female, Human Variation Panel), and universally methylated male gDNA (Serologicals, Norcross, Ga.). After bisulfite conversion, PCR was performed as described above, using the FMR1 specific PCR-primer pairs presented in Table 1.

### Results

Amplicons were selected in the CpG islands of promoter sequences of genes involved in various cancers, and often overlapped with regions previously reported in the literature to be methylated in cancer patients. Primers for the bisulfite-converted gDNA shown in Table 1 were designed to amplify a region regardless of methylation state by annealing to non-CpG sequences that flank regions of high CpG-content. An amplicon from fully UnMe gDNA will contain no C's (in the forward strand) while an amplicon from a fully Me gDNA will contain “C” at all CpG motifs. The cumulative effect of multiple C/T differences in the amplicons results in a mass/charge difference. Results show that the amplicons from Me and UnMe gDNA are resolved by CE, with the forward strand of the amplicon derived from Me gDNA migrating faster than UnMe counterpart.

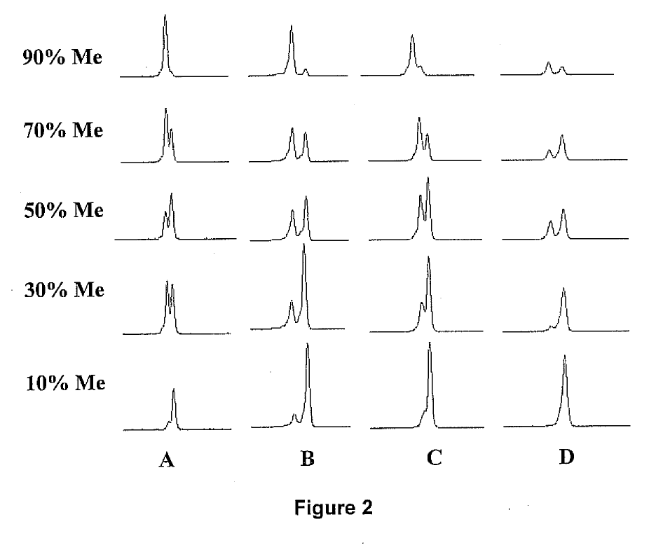

FIG. 2 graphically depicts typical electropherograms for four amplicons generated from known ratios of methylated and unmethylated gDNA template, following bisulfite conversion. The four amplicons (of the 18 shown in summary in Table 2 below) investigated by MDFS are shown and vary in size and number of CpG dinucleotides as follows: A. RasSF (223 nt, 16 CpG); B. p. 16 (284 nt, 28 CpG); C. APC (285 nt, 22 CpG); D. Dapk (416 nt, 39 CpG). The percentage of methylation is indicated for each row. PCR bias favoring the unmethylated amplicon is clearly evident for the largest amplicon (D, Dapk). The best resolution between the amplicons from fully methylated and fully unmethylated gDNA was observed for amplicons that were ˜10% CpG or greater, and were 200 or more nucleotides long. Shorter amplicons, or a lower percentage of CpG, or both, results in more similar migration times so that some amplicons were not always base-line resolved.

### Prediction of Electrophoresis Migration Times and Amplicon Size

The differences in migration time of the PCR products arising from Me and UnMe gDNA are due to differences in the electrophoretic mobility of the individual nucleotides that make up gDNA. Migration times of oligonucleotides in capillary electrophoresis under denaturing conditions and in a sieving medias, can be modeled as linearly related to the number of nucleotides N and expressed as the relationship:

t_mig+k+nN

where k is a constant offset and n is a coefficient relating migration time and size. Both coefficients k and n can be determined by least-squares analysis of a set of experiments measuring the migration times of oligonucleotides with known lengths.

In the model above, while oligonucleotide size is linearly related to migration time, it is also known that base composition, the number of A-, G-, T-, and C-residues in a single-stranded DNA (ssDNA) fragment affects electrophoretic mobility and hence migration time. Typically, two ssDNA fragments with identical length N will exhibit different migration times due to differences in nucleotide composition.

Accordingly, migration times can also be correlated to oligonucleotide composition (see T. Satow, T. Akiyama, A. Machida, Y. Utagawa, and H. Kobayashi, Simultaneous determination of the migration coefficient of each base in heterologous oligo-DNA by gel-filled capillary electrophoresis, J Chrom 652 (1993) 23-30). If a linear model is used, a variable can be assigned to each nucleotide. For example, the equation described by Satow is

t_{—}mig=k+aA+gG+tT+cC

where A, G, T and C are the numbers of nucleotides present in the ssDNA. In other words (N=A+G+T+C) and a, g, t, and c are the base-specific coefficients.

Migration time measurements for a set of five or more (determined or over-determined systems) oligonucleotides with known base composition will allow calculation of the coefficients (k, a, g, t and c) under specific experimental separation conditions. The coefficients can then in turn be used to calculate a predicted migration time of any oligonucleotide given the composition under the same separation conditions. One skilled in the art will appreciated that other methods numerical methods can be used to determine model coefficients. For example, neural network or machine learning methods can be employed. In addition, more complex models can be used such as non-linear models or models that account for variation in migration times due to context-dependant effects. Context-dependant effects can occur when, for example, homopolymer runs occur or specific subsequences within the oligonucleotide result in migration time variation.

In order to obtain better precision of the regression analysis, various embodiments replace migration time as the independent variable by the fragment size as determined using size standards in conjunction with the oligonucleotide and analysis by GeneScan software (Applied Biosystems). Using size as the independent variable can result in equations relating the ssDNA fragment size to the length of the oligonucleotide or its composition. These equations appear as

size=k+nN

size=aA+gG+tT+cC

A set of 50 synthetic oligonucleotides ranging from 19-61 nts, many with single nucleotide differences, was used to determine values for the mobility differences. The set is found in Table 2 below and is utilized as the learning set.

| | | | | | | | | Difference |
|---|---|---|---|---|---|---|---|---|
| Obser- | | | | | | Observed | Predicted | Pred.-Ob. |
| vation # | A | G | T | C | N | Size nt. | Size nt. * | nt |
| 1 | 7 | 5 | 5 | 2 | 19 | 22.76 | 21.86 | −0.90 |
| 2 | 7 | 5 | 4 | 3 | 19 | 21.52 | 21.76 | 0.24 |
| 3 | 10 | 5 | 9 | 7 | 31 | 31.68 | 32.04 | 0.36 |
| 4 | 10 | 5 | 8 | 8 | 31 | 30.5 | 31.94 | 1.44 |
| 5 | 8 | 7 | 15 | 7 | 37 | 38.83 | 38.26 | −0.57 |
| 6 | 8 | 7 | 14 | 8 | 37 | 37.93 | 38.16 | 0.23 |
| 7 | 12 | 8 | 13 | 10 | 43 | 43.99 | 43.33 | −0.66 |
| 8 | 11 | 9 | 13 | 10 | 43 | 43.2 | 43.69 | 0.49 |
| 9 | 13 | 10 | 15 | 11 | 49 | 48.55 | 49.16 | 0.61 |
| 10 | 13 | 9 | 16 | 11 | 49 | 49.85 | 48.89 | −0.96 |
| 11 | 13 | 9 | 15 | 12 | 49 | 48.89 | 48.79 | −0.10 |
| 12 | 5 | 3 | 7 | 4 | 19 | 23.51 | 21.31 | −2.20 |
| 13 | 5 | 3 | 6 | 5 | 19 | 22.11 | 21.21 | −0.90 |
| 14 | 5 | 3 | 7 | 10 | 25 | 26.92 | 26.18 | −0.74 |
| 15 | 5 | 3 | 6 | 11 | 25 | 25.49 | 26.08 | 0.59 |
| 16 | 12 | 4 | 8 | 7 | 31 | 31.76 | 31.58 | −0.18 |
| 17 | 12 | 4 | 7 | 8 | 31 | 30.97 | 31.48 | 0.51 |
| 18 | 9 | 9 | 8 | 11 | 37 | 37.21 | 38.29 | 1.08 |
| 19 | 9 | 8 | 9 | 11 | 37 | 38.63 | 38.02 | −0.61 |
| 20 | 14 | 11 | 9 | 9 | 43 | 44.56 | 44.04 | −0.52 |
| 21 | 13 | 11 | 10 | 9 | 43 | 45.7 | 44.14 | −1.56 |
| 22 | 13 | 11 | 9 | 10 | 43 | 43.71 | 44.03 | 0.32 |
| 23 | 13 | 14 | 11 | 11 | 49 | 50.53 | 50.23 | −0.30 |
| 24 | 13 | 14 | 10 | 12 | 49 | 49.9 | 50.12 | 0.22 |
| 25 | 7 | 7 | 3 | 2 | 19 | 24.72 | 22.40 | −2.32 |
| 26 | 6 | 8 | 3 | 2 | 19 | 22.03 | 22.76 | 0.73 |
| 27 | 9 | 8 | 5 | 3 | 25 | 28.29 | 27.86 | −0.43 |
| 28 | 9 | 8 | 4 | 4 | 25 | 27.45 | 27.76 | 0.31 |
| 29 | 11 | 9 | 7 | 4 | 31 | 31.7 | 33.33 | 1.63 |
| 30 | 11 | 8 | 8 | 4 | 31 | 33.11 | 33.06 | −0.05 |
| 31 | 12 | 7 | 8 | 10 | 37 | 37.67 | 37.57 | −0.10 |
| 32 | 11 | 8 | 8 | 10 | 37 | 36.59 | 37.93 | 1.34 |
| 33 | 15 | 7 | 11 | 10 | 43 | 44.08 | 42.77 | −1.31 |
| 34 | 14 | 8 | 11 | 10 | 43 | 43.02 | 43.13 | 0.11 |
| 35 | 18 | 11 | 8 | 12 | 49 | 48.44 | 48.84 | 0.40 |
| 36 | 18 | 10 | 8 | 13 | 49 | 48.58 | 48.47 | −0.11 |
| 37 | 6 | 3 | 7 | 3 | 19 | 19.77 | 21.32 | 1.55 |
| 38 | 6 | 2 | 7 | 4 | 19 | 20.22 | 20.95 | 0.73 |
| 39 | 6 | 9 | 5 | 5 | 25 | 28.36 | 28.21 | −0.15 |
| 40 | 5 | 9 | 6 | 5 | 25 | 27.76 | 28.31 | 0.55 |
| 41 | 7 | 9 | 8 | 7 | 31 | 34 | 33.40 | −0.60 |
| 42 | 7 | 9 | 7 | 3 | 31 | 32.85 | 33.30 | 0.45 |
| 43 | 5 | 3 | 5 | 8 | 21 | 23.71 | 22.73 | −0.98 |
| 44 | 13 | 3 | 6 | 6 | 28 | 28.6 | 28.57 | −0.03 |
| 45 | 12 | 4 | 6 | 6 | 28 | 27.8 | 28.94 | 1.14 |
| 46 | 3 | 5 | 24 | 5 | 37 | 36.62 | 38.42 | 1.80 |
| 47 | 7 | 5 | 31 | 2 | 45 | 45.17 | 45.67 | 0.50 |
| 48 | 8 | 3 | 37 | 5 | 53 | 52.65 | 52.06 | −0.59 |
| 49 | 8 | 3 | 36 | 6 | 53 | 51.92 | 51.95 | 0.03 |
| 50 | 4 | 5 | 45 | 7 | 61 | 60.56 | 60.09 | −0.47 |

* Predicted size after regression analysis by composition.

The observed size of each oligonucleotide relative to a size standard was obtained under the analysis conditions described in the above Materials and Methods. In particular, a linear least-squares regression analysis was performed on the 50-oligonucleotide data set by length as well as by composition. The size was correlated to the number of each of the nucleotide bases (A, G, T, C) in the oligonucleotide. The mobility coefficient for each base (a, g, t, c) could then be determined.

TABLE III
Regression analysis of Table II

| | Length (N) | Composition |
|---|---|---|
| | vs. Size | (A, G, T, C) |
| Coefficient | Observed | vs. size observed |
| k | 4.427 | 4.010 |
| a | — | 0.819 |
| g | — | 1.180 |
| t | — | 0.916 |
| c | — | 0.812 |
| n | 0.914 | — |
| Standard Deviation σ (nt.) | ±1.1355 | ±0.936 |
| R² | 0.988 | 0.993 |

The solved equation provides a linear relationship (correlation coefficient R²=0.993) between base composition and the apparent fragment size. The coefficients for the relevant bases, C and T, are c=0.812 and t=0.916. In the context of bisulfite methylation analysis, a single C/T transition results in an approximate 0.1-nt difference in the apparent size of the fragment, and the observed difference is additive and linear as multiple instances of C/T mutations occur in the PCR product. As the value of the coefficient suggests, C-containing product resulting from Me gDNA will always migrate as the apparently shorter fragment compared to the PCR-product from UnMe gDNA. This finding can help in assignment of peaks in instances of mixed methylation status. The larger amplicons deviate from the prediction of 0.1 nt per C/T transition, resulting in an enhancement of the observed mobility differences, possibly due to single-stranded secondary structure differences. Again, one skilled in the art will appreciate that more complex models can be used, for example models that incorporate secondary structure can be developed and either trained using a numerical or machine learning approach or secondary structure predictors such as Mfold (see Mfold web server for nucleic acid folding and hybridization prediction. Nucleic Acids Res. 31 (13), 3406-15, (2003)) can be used or other such tools. The additional computational cost of training more complex models and/or using larger datasets can be offset by the further enhanced prediction algorithms.

The resulting coefficients obtained from Table II are applied to the panel of 18 genes subjected to methylation detection analysis as described herein. A bisulfite treated and an untreated sample were generated from the gDNA and the resulting DNA was PCR amplified in 18 different loci and subjected to CE analysis. The resulting experimentally observed size for each of the two products as well as the observed size difference are listed in Table IV. Using the known sequence of the amplicon and the presumed sequence of an amplicon arising from a per-methylated gDNA, a predicted size is calculated for each of the two products, and the result is listed in Table IV below.

TABLE IV

| | | | Observed size, nt. | | | Predicted size, nt. | | |
|---|---|---|---|---|---|---|---|---|
| | | Amplicon | Methylated | Unmethylated | | Methylated | Unmethylated | |
| Gene | # CpG | length, nt. | DNA | DNA | Difference | DNA. | DNA | Difference |
| BRCA | 11 | 186 | 186.7 | 187.9 | 1.2 | 179.7 | 180.9 | 1.2 |
| Srbc | 13 | 187 | 188.7 | 189.7 | 1 | 183.4 | 184.8 | 1.4 |
| TIMP | 15 | 208 | 212.11 | 213.28 | 1.17 | 207.2 | 208.8 | 1.6 |
| CDH1 | 14 | 209 | 214.4 | 215.8 | 1.4 | 211.9 | 213.3 | 1.4 |
| MYOD1 | 9 | 222 | 225.98 | 225.98 | 0 | 212.6 | 213.5 | 0.9 |
| RasSF(187) | 16 | 223 | 229.24 | 230.45 | 1.21 | 224 | 225.6 | 1.6 |
| FMR1 | 22 | 225 | 225.8 | 230.66 | 4.86 | 219.5 | 221.8 | 2.3 |
| MGMT | 11 | 264 | 268.58 | 268.58 | 0 | 264.3 | 265.5 | 1.2 |
| APC | 22 | 285 | 290.36 | 291.87 | 1.51 | 289.2 | 291.5 | 2.3 |
| p16 | 28 | 287 | 296.1 | 298.7 | 2.6 | 294.7 | 297.6 | 2.9 |
| ER | 26 | 305 | 309.87 | 311.96 | 2.09 | 299.1 | 301.8 | 2.7 |
| MLH1 | 30 | 310 | 314.38 | 317.33 | 2.95 | 302.8 | 305.9 | 3.1 |
| ALX3 | 34 | 311 | 312.4 | 319.7 | 7.3 | 308.8 | 312.3 | 3.5 |
| p15 | 30 | 326 | 328.5 | 333.8 | 5.3 | 327.5 | 330.6 | 3.1 |
| COX2 | 25 | 328 | 332.82 | 335.25 | 2.43 | 318.7 | 321.3 | 2.6 |
| DAPk | 39 | 416 | 420.1 | 422.9 | 2.8 | 413.5 | 417.6 | 4.1 |
| RB1 | 60 | 446 | 444.88 | 452.47 | 7.59 | 437.2 | 443.5 | 6.3 |
| RasSF (451) | 52 | 487 | 486.09 | 494.67 | 8.58 | 478.9 | 484.3 | 5.4 |

As seen in Table IV, the panel of 18 gene regions showing the observed and predicted sizes of the amplicons generated from methylated and unmethylated gDNA followed by CE analysis. The data is listed in order of increasing amplicon length. Longer amplicons and amplicons that are CpG-rich had the greatest degree of separation, while shorter amplicons and lower CpG percentage had poorer separation. Amplicon separation was enhanced when POP 6™ polymer or reduced temperature (55° C. instead of 60° C.) was used for CE. The results were obtained using POP 4™ polymer at 60° C., as described in Materials and Methods.

In various embodiments, and as illustrated in the tables, an observed difference in size is compared to the predicted difference in size of the amplicons. There is a particularly close correlation for small amplicons with a limited number of dCpG's. One of the factors contributing to differences between observed size and predicted size differences is that the larger amplicons have a higher number of dCpG's with secondary structure formation in the amplicon. The prediction algorithm does not take this into account, but could be accommodated and thereby offset with a larger data (learning) set.

Efficient unbiased PCR amplification from Me and UnMe gDNA is essential for any PCR amplification-dependent method designed to detect methylation following bisulfite conversion. Amplicons generated from methylated gDNA remain CpG-rich relative to amplicons from unmethylated gDNA, and are often amplified less efficiently, although amplification bias may favor either amplicon. The forward primer for PCR is usually very “T” rich and the reverse primer is “A” rich resulting in an increased incident of primer-dimer (see Q. Chou, M. Russell, D. E. Birch, J. Raymond, and W. Bloch, Prevention of pre-PCR mis-priming and primer dimerization improves low-copy-number amplifications, Nucleic Acids Res 20 (1992) 1717-1723) and secondary amplicon formation, which can further reduce efficient amplification of the intended target. Sequence specificity for a primer composed of all four bases requires a length of about 18-22 nts, but increases to an estimated 28 nts for a primer containing only 3 of the 4 bases. Because the sense and antisense strands are no longer self-complementary, a “first strand” synthesis from bisulfite-converted gDNA may be needed prior to exponential amplification. The polymerase used for first strand synthesis is ideally capable of “reading” U (and 5mC) in the template.

Amplicons from bisulfite-converted gDNA often have homopolymer stretches of ≧9 T's (A's) which may result in poor (or no) amplification. A broadened signal is observed during electrophoresis for these amplicons due to enzyme “slippage” causing n+1 and n−1 sequences. Heuristics rules based on observation can be used to improve PCR results. For example by selecting amplicons containing no greater than 8 consecutive Ts (or As), exemplary PCR reactions were nearly always successful. Optimized conditions are further described in the following paragraph. Achieving these two requirements, i.e., avoidance of homopolymer stretches and designing appropriately long primers in a non-CpG region, may restrict the number of primers that can be used for an amplicon within a CpG island; perhaps to a single primer. However, in some instances, since the sense and antisense strands are no longer complementary, it is sometimes possible to select suitable amplicons for the described fragment analysis method from the corresponding antisense strand.

### Tailed Primers

The aforementioned restrictive requirements for selecting an appropriate amplicon and primer pair can impose several challenges for achieving successful PCR. In spite of the complementary forward and reverse primers and presence of multiple sequence matches to the bisulfite-converted gDNA, the PCR conditions described above in “Materials and Methods” were highly successful. Primers were tailed with the −21M13 sequence which provided several benefits. After an initial cycle (empirically 2-5 are needed) a tailed amplicon is formed and created a lengthened primer-binding site with an increased Tₘ. The tailed portion has all four bases in the sequence, which increased the specificity, after the initial formation of the amplicon, relative to the rest of the bisulfite-converted genome. Subsequent PCR at a higher annealing temperature resulted in higher specificity for the targeted amplicon, and reduced both primer-dimer and secondary amplicon formation. A shorter tailed sequence could also be used, but incorporation of the −21M13 tails provided a universal primer binding site suitable for any (additional) downstream analysis methods.

A decrease in PCR bias of the methylated and unmethylated samples is observed using tailed primers and the presently described thermocycling conditions. Some PCR bias was still observed, especially for the longer amplicons.

Incorporation of Modified dCTP

The electrophoretic migration of an amplicon can be influenced by incorporation of modified dNTP(s) during PCR. Livak et al. (see Q. Chou, M. Russell, D. E. Birch, J. Raymond, and W. Bloch, Prevention of pre-PCR mis-priming and primer dimerization improves low-copy-number amplifications, Nucleic Acids Res 20 (1992) 1717-1723) reported a very large “drag” effect on the migration of amplicons with just a single nucleotide polymorphism (SNP) following incorporation of a biotinylated C residue wherein the binding moiety was tethered to a 36-carbon linkage, namely, biotin-aha-dCTP. This ability of biotin-aha-dCTP to resolve one SNP is quite remarkable considering that bands on a slab gel were used for analysis rather than CE, which can generally give much higher resolution. Fragments from gDNA of mixed methylation states that are poorly separated due to the presence of only a few CpG's can, in principle, be resolved when a modified C is incorporated.

### Exemplary Analysis of Methylation in the Fragile-X FMR1 Gene

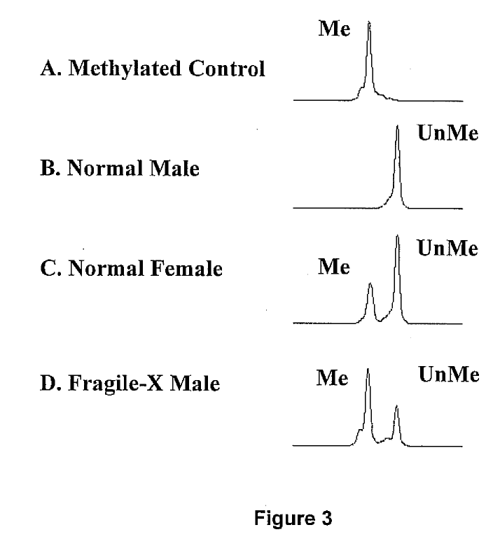

Methylation in the sequence upstream of the expanded CGG repeat has been reported in individuals with fragile-X syndrome. The practical utility of methylation detection by direct fragment analysis after PCR was demonstrated on gDNA samples isolated from immortalized cell lines obtained from Coriell. The amplicon region selected was that previously found to be methylated in fragile-X patients. A comparison of the PCR results from bisulfite-converted gDNA from a control male, control female, universally methylated male, and a fragile-X male is shown in FIG. 3.

The methylation status of the FMR1 gene in the four individual gDNA samples, after bisulfite conversion, was determined by MDFS analysis and the results are: A. methylated control gDNA, showing only the amplicon from methylated gDNA; B. control male gDNA, whereon only the amplicon from unmethylated gDNA is seen; C. control female gDNA, which has amplicons for both methylated and unmethylated gDNA due to X-chromosome silencing; and D. fragile-X male gDNA, which has amplicons for both methylated and unmethylated gDNA, in contrast to control male gDNA where only unmethylated gDNA is detected.

These results are consistent with the expectations discussed above. One of the X-chromosomes in female gDNA is normally silenced by methylation, and an equal mix of methylation states should be present. Due to amplification bias, the signal for the unmethylated amplicon is larger than the methylated signal. The control male DNA has no methylation, while the fragile-X male DNA has a large amount of methylation, exceeding that detected in the control female DNA. The use of standard curves to account for PCR bias could allow for more accurate determination of methylation. Determination of methylation in fragile-X female DNA will be more difficult due to the inherent presence of methylation on the silenced chromosome.

Accordingly, the high-resolution capability of CE based on mass/charge for a given DNA fragment has been employed to separate amplicons having multiple C vs. T polymorphic sites. Although similarly obtained, data for G vs. A is not presented herein. This analytical method, termed Methylation Dependent Fragment Separation (MDFS), is based on finding that CE separation quantitatively correlates with mass/charge differences, namely, empirically predicted vs. experimentally observed earlier migration of the lower mass (methylated) amplicon when analyzing the forward strand. C is 15 atomic mass units less than T. and the cumulative number of C vs. T sites in one amplicon thus results in a significant overall mass/charge difference. The above-described algorithm to calculate the mobility differences of the amplicons from Me and UnMe gDNA agreed with the actual observed separation of roughly 0.1-nt difference per polymorphic site for the shorter amplicons.

In the exemplary embodiments, bisulfite treatment must occur prior to PCR, since currently available polymerases do not discriminate between 5mC and C to a significant extent (although very small differences have been observed by direct sequencing of gDNA (see A. Bart, M. W. van Passel, K. van Amsterdam, and A. van der Ende, Direct detection of methylation in genomic DNA, Nucleic Acids Res 33 (2005) e124)). Bisulfite-converted PCR amplicons are used in techniques such as single-base extension (e.g., SNaPshot® kit) as described in K. Uhlmann, A. Brinckmann, M. R. Toliat, H. Ritter, and P. Nurnberg, Evaluation of a potential epigenetic biomarker by quantitative methyl-single nucleotide polymorphism analysis, Electrophoresis 23 (2002) 4072-4079; and Z. A. Kaminsky, A. Assadzadeh, J. Flanagan, and A. Petronis, Single nucleotide extension technology for quantitative site-specific evaluation of metC/C in GC-rich regions, Nucleic Acids Res 33 (2005) e95, bisulfite sequencing as described in M. Frommer, L. E. McDonald, D. S. Millar, C. M. Collis, F. Watt, G. W. Grigg, P. L. Molloy, and C. L. Paul, A genomic sequencing protocol that yields a positive display of 5-methylcytosine residues in individual DNA strands, Proc Natl Acad Sci USA 89 (1992) 1827-1831, cloning and sequencing, melting curve analysis as described in J. Worm, A. Aggerholm, and P. Guldberg, In-tube DNA methylation profiling by fluorescence melting curve analysis, Clin Chem 47 (2001) 1183-1189; and D. T. Akey, J. M. Akey, K. Zhang, and L. Jin, Assaying DNA methylation based on high-throughput melting curve approaches, Genomics 80 (2002) 376-384, combined bisulfite restriction analysis (COBRA) (see Z. Xiong, and P. W. Laird, COBRA: a sensitive and quantitative DNA methylation assay, Nucleic Acids Res 25 (1997) 2532-2534), and single-strand conformational polymorphism (SSCP) (see N. Burri, and P. Chaubert, Complex methylation patterns analyzed by single-strand conformation polymorphism, Biotechniques 26 (1999) 232-234). Direct analysis of the amplicon from bisulfite-converted gDNA without any additional sample processing steps provides a relatively simple analytical tool for detecting the presence of methylation. Moreover, the same amplicon(s) can then be used for additional analyses that confirm/strengthen the results. Additional sample processing may introduce bias that distorts quantitative measurements and requires extra time and cost. In the event that CE is unable to resolve the methylated and unmethylated amplicons, or if the results suggest the presence of variable methylation in the amplicon, bisulfite sequencing or other techniques can still be applied to the amplicon without having created any extra steps (other than the CE analysis).

Accordingly, the exemplary embodiments herein have provided further improvements to earlier described protocols for bisulfite conversion, as well as procedures for improving the success of PCR amplification despite limited primer selection, and a relatively simple and novel CE method for analysis of methylated gDNA. Improved bisulfite sequencing of amplicons that were generated is described herein, and use both the FAM dye-labeled or unlabeled amplicons as templates for direct sequencing by means of conventional sequencing instruments that employ the Applied Biosystems KB™ Basecaller. The presently described MDFS analytical method can be used in combination with other analysis techniques, or serve as a fast screening tool to determine methylation ratios.

Various other exemplary embodiments may provide mechanisms for data analysis utilizing the exemplary methylation detection analysis. Non-limiting examples include predicting the observed size given the gDNA sequence (length and composition of the amplicon) for both fragments (treated and untreated) and hence a difference and migration order. Further, given experimentally observed difference in size between the treated and untreated DNA, the algorithms allow derivation of the number of methylated dCpG's from the experimental data. Even further, the present teachings can embody a software tool for the detection of methylation by CE analysis of PCR products of bisulfite treated template DNA. They can also embody a software tool for the design of oligonucleotides containing mobility modifiers. The tool will allow prediction of the effect of mobility modifying bases on migration behavior, especially for the development of multiplex sets (multiplex PCR products as used in human identification (HID), Snapshot and Snplex genotyping oligonucleotide sets). Once the coefficient for the modified based has been established, mobility effects of the modifiers can be predicted in-silico during oligonucleotide design.

### Computer System Implementation

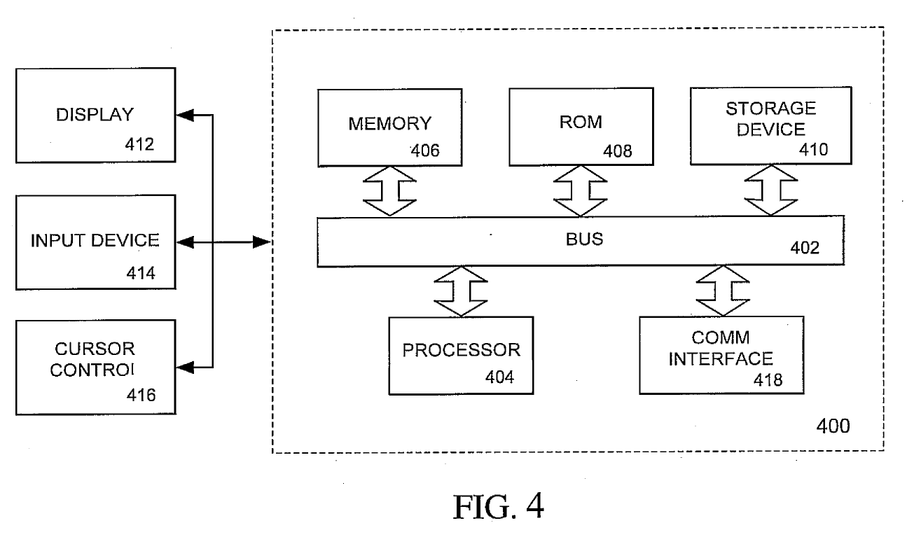

FIG. 4 is a block diagram that illustrates a computer system 400, upon which embodiments of the present teachings may be implemented. Computer system 400 includes a bus 402 or other communication mechanism for communicating information, and a processor 404 coupled with bus 402 for processing information. Computer system 400 also includes a memory 406, which can be a random access memory (RAM) or other dynamic storage device, coupled to bus 402, and instructions to be executed by processor 404. Memory 406 also may be used for storing temporary variables or other intermediate information during execution of instructions to be executed by processor 404. Computer system 400 further includes a read only memory (ROM) 408 or other static storage device coupled to bus 402 for storing static information and instructions for processor 404, A storage device 410, such as a magnetic disk or optical disk, is provided and coupled to bus 402 for storing information and instructions.

Computer system 400 may be coupled via bus 402 to a display 412, such as a cathode ray tube (CRT) or liquid crystal display (LCD), for displaying information to a computer user. An input device 414, including alphanumeric and other keys, is coupled to bus 402 for communicating information and command selections to processor 404. Another type of user input device is cursor control 416, such as a mouse, a trackball or cursor direction keys for communicating direction information and command selections to processor 404 and for controlling cursor movement on display 412. This input device typically has two degrees of freedom in two axes, a first axis (e.g., x) and a second axis (e.g., y), that allows the device to specify positions in a plane.

Consistent with certain embodiments of the present teachings, functions including methylation prediction, training of predictors, analysis of electrophoresis data, printing, storage and presentation of results, and interactive display of results can be performed by computer system 400 in response to processor 404 executing one or more sequences of one or more instructions contained in memory 406. Such instructions may be read into memory 406 from another computer-readable medium, such as storage device 410. Execution of the sequences of instructions contained in memory 406 causes processor 404 to perform the process states described herein. Alternatively hard-wired circuitry may be used in place of or in combination with software instructions to implement the invention. Thus implementations of the present teachings are not limited to any specific combination of hardware circuitry and software.

The term “computer-readable medium” as used herein refers to any media that participates in providing instructions to processor 404 for execution. Such a medium may take many forms, including but not limited to, non-volatile media, volatile media, and transmission media. Non-volatile media includes, for example, optical or magnetic disks, such as storage device 410. Volatile media includes dynamic memory, such as memory 406. Transmission media includes coaxial cables, copper wire, and fiber optics, including the wires that comprise bus 402. Transmission media can also take the form of acoustic or light waves, such as those generated during radio-wave and infra-red data communications.

The present teachings provide a variety of structural arrangements, techniques, and/or methodology useful for methylation prediction. It should be understood that although in some cases the embodiments described herein may focus on a particular aspect, various embodiments may be combined to form a system and/or substrate configuration useful for methylation prediction. The various embodiments described herein are not intended to be mutually exclusive.

For the purposes of this specification and appended claims, unless otherwise indicated, all numbers expressing quantities, percentages or proportions, and other numerical values used in the specification and claims, are to be understood as being modified in all instances by the term “about.” Accordingly, unless indicated to the contrary, the numerical parameters set forth in the following specification and attached claims are approximations that may vary depending upon the desired properties sought to be obtained by the present invention. At the very least, and not as an attempt to limit the application of the doctrine of equivalents to the scope of the claims, each numerical parameter should at least be construed in light of the number of reported significant digits and by applying ordinary rounding techniques.

Notwithstanding that the numerical ranges and parameters setting forth the broad scope of the invention are approximations, the numerical values set forth in the specific examples are reported as precisely as possible. Any numerical value, however, inherently contains certain errors necessarily resulting from the standard deviation found in their respective testing measurements. Moreover, all ranges disclosed herein are to be understood to encompass any and all subranges subsumed therein. For example, a range of “less than 10” includes any and all subranges between (and including) the minimum value of zero and the maximum value of 10, that is, any and all subranges having a minimum value of equal to or greater than zero and a maximum value of equal to or less than 10, e.g., 1 to 5.

It is noted that, as used in this specification and the appended claims, the singular forms “a,” “an,” and “the” include plural referents unless expressly and unequivocally limited to one referent. Thus, for example, reference to “a layer” includes two or more different layers. As used herein, the term “include” and its grammatical variants are intended to be non-limiting, such that recitation of items in a list is not to the exclusion of other like items that can be substituted or added to the listed items.

Various embodiments of the teachings are described herein. The teachings are not limited to the specific embodiments described, but encompass equivalent features and methods as known to one of ordinary skill in the art. Other embodiments will be apparent to those skilled in the art from consideration of the present specification and practice of the teachings disclosed herein. It is intended that the present specification and examples be considered as exemplary only.

1. A method for predicting an amount of methylation of at least one target region, the method comprising:
establishing an observed size of a plurality of oligonucleotides relative to a size standard;
correlating the observed size to a number of each of nucleotide base in each of the plurality of oligonucleotides;
determining a mobility coefficient for each base of each respective oligonucleotide;
applying determined mobility coefficients to a predetermined number of polynucleotides subjected to methylation detection analysis;
treating said plurality of oligonucleotides with a modifying agent to obtain amplicons in methylated and unmethylated target regions;
distinguishing amplicons derived from methylated and unmethylated target regions based on their relative mobilities; and
predicting the degree of methylation of distinguished methylated regions.

2. The method of claim 1, further comprising calculating a predicted size of each of the predetermined number of subjected polynucleotides.

3. The method of claim 2, wherein calculating the predicted size includes using a known sequence of the amplicon and presumed sequence of the amplicon arising from a per-methylated dDNA, and calculating the predicted size for each of the two products.

4. The method of claim 1, wherein establishing an observed size of a plurality of oligonucleotides relative to a size standard includes providing a predetermined panel of oligonucleotides as a learning data set, and measuring a size of each of the plurality of oligonucleotides by capillary electrophoresis using the size standard.

5. The method of claim 1, wherein the observed size is related to a length of a corresponding oligonucleotide.

6. The method of claim 1, wherein the observed size is related to a composition of a corresponding oligonucleotide.

7. The method of claim 1, wherein correlating the observed size to the number of each of the nucleotide bases in each of the plurality of oligonucleotides includes at least the equation: size=a A+g G+t T+c C, where A, G, T and C are the numbers of nucleotides present in a single strand DNA, and a, g, t, and c are base-specific mobility coefficients.

8. The method of claim 7, wherein observed size for a set of at least five oligonucleotides with known base compositions enables calculation of the base-specific mobility coefficients under predetermined separation conditions, where the coefficients are used to calculate the predicted size of any oligonucleotide having the known base composition under the same separation conditions.

9. The method of claim 4, wherein determining coefficients of oligonucleotide compositions from the learning data set comprises:
performing a regression analysis on the data set using length of oligonucleotide; and
performing a regression analysis on the data set using composition of oligonucleotide.

10. The method of claim 4, wherein the learning set includes a set of 50 synthetic oligonucleotides from about 19 to about 61 nts.

11. The method of claim 1, wherein the modifying agent includes sodium bisulfite.

12. A method for predicting a size of amplicons generated from methylated and unmethylated gDNA, the method comprising:
establishing an observed size of a plurality of oligonucleotides relative to a size standard;
correlating the observed size to a number of each of the nucleotide bases in each of the plurality of oligonucleotides;
determining a mobility coefficient for each base of each respective oligonucleotide;
applying determined mobility coefficients to a predetermined number of polynucleotides subjected to methylation detection analysis; and
calculating the predicted size of the amplicons using the determined mobility coefficients and a known sequence of the amplicon and a presumed sequence of an amplicon arising from a per-methylated gDNA.

13. The method of claim 12, wherein establishing an observed size of a plurality of oligonucleotides relative to a size standard includes providing a predetermined panel of oligonucleotides as a learning data set, and measuring a size of each of the plurality of oligonucleotides by capillary electrophoresis using the size standard.

14. The method of claim 12, wherein the observed size is related to a length of a corresponding oligonucleotide.

15. The method of claim 12, wherein the observed size is related to a composition of a corresponding oligonucleotide.

16. The method of claim 12, wherein correlating the observed size to the number of each of the nucleotide bases in each of the plurality of oligonucleotides includes at least the equation: size=a A+g G+t T+c C, where A, G, T and C are the numbers of nucleotides present in a single strand DNA, and a, g, t, and c are base-specific mobility coefficients.

17. The method of claim 16, wherein observed size for a set of at least five oligonucleotides with known base compositions enables calculation of the base-specific mobility coefficients under predetermined separation conditions, where the coefficients are used to calculate the predicted size of any oligonucleotide having the known base composition under the same separation conditions.

18. The method of claim 13, wherein determining coefficients of oligonucleotide compositions from the learning data set comprises:
performing a regression analysis on the data set using length of oligonucleotide; and
performing a regression analysis on the data set using composition of oligonucleotide.

19. The method of claim 13, wherein the learning set includes a set of 50 synthetic oligonucleotides from about 19 to about 61 nts.

20. The method of claim 12, wherein the modifying agent includes sodium bisulfite.

21. A method of calculating a predicted size for an untreated (DNA) product and a bisulfite treated (DNA) product comprising:
providing a known sequence of an amplicon and a presumed sequence of an amplicon arising from a per-methylated gDNA;
calculating a DNA fragment size to a length of the corresponding oligonucleotide; and
calculating a DNA fragment size to a composition of the corresponding oligonucleotide.


SEQUENCE LISTING (36 sequences)

SEQ ID NO: 1 - DNA, 45 bp, Artificial Sequence
  Description of Artificial Sequence Synthetic primer
tgtaaaacgacggccagtatttgagaaattttatagtttgttttt

SEQ ID NO: 2 - DNA, 43 bp, Artificial Sequence
  Description of Artificial Sequence Synthetic primer
gcaggaaacagctatgacctattctaaaaaactactacttaac

SEQ ID NO: 3 - DNA, 43 bp, Artificial Sequence
  Description of Artificial Sequence Synthetic primer
tgtaaaacgacggccagttggggttaataggttttttagtagg

SEQ ID NO: 4 - DNA, 44 bp, Artificial Sequence
  Description of Artificial Sequence Synthetic primer
gcaggaaacagctatgaccaactccaactataactcaaacaaac

SEQ ID NO: 5 - DNA, 44 bp, Artificial Sequence
  Description of Artificial Sequence Synthetic primer
gtgtaaaacgacggccagttggtttgggttagagatatttagtg

SEQ ID NO: 6 - DNA, 45 bp, Artificial Sequence
  Description of Artificial Sequence Synthetic primer
gcaggaaacagctatgaccttcaaatccttataaaaaataatacc

SEQ ID NO: 7 - DNA, 45 bp, Artificial Sequence
  Description of Artificial Sequence Synthetic primer
tgtaaaacgacggccagttttagtaattttaggttagagggttat

SEQ ID NO: 8 - DNA, 41 bp, Artificial Sequence
  Description of Artificial Sequence Synthetic primer
gcaggaaacagctatgacctaactacaaccaaataaacccc

SEQ ID NO: 9 - DNA, 47 bp, Artificial Sequence
  Description of Artificial Sequence Synthetic primer
gtgtaaaacgacggccagtttttgtgtttttaatgttttgttttttt

SEQ ID NO: 10 - DNA, 41 bp, Artificial Sequence
  Description of Artificial Sequence Synthetic primer
gcaggaaacagctatgacccctttccaaacctctccaacac

SEQ ID NO: 11 - DNA, 43 bp, Artificial Sequence
  Description of Artificial Sequence Synthetic primer
gtgtaaaacgacggccagttagtttaatgagtttaggtttttt

SEQ ID NO: 12 - DNA, 39 bp, Artificial Sequence
  Description of Artificial Sequence Synthetic primer
gcaggaaacagctatgaccctacacccaaatttccatta

SEQ ID NO: 13 - DNA, 42 bp, Artificial Sequence
  Description of Artificial Sequence Synthetic primer
tgtaaaacgacggccagttgagtgtatttttgtagaaatggg

SEQ ID NO: 14 - DNA, 43 bp, Artificial Sequence
  Description of Artificial Sequence Synthetic primer
gcaggaaacagctatgacctctctcttcaaataacctaaaaac

SEQ ID NO: 15 - DNA, 44 bp, Artificial Sequence
  Description of Artificial Sequence Synthetic primer
gtgtaaaacgacggccagtatggtttttggtttatgaaggttat

SEQ ID NO: 16 - DNA, 46 bp, Artificial Sequence
  Description of Artificial Sequence Synthetic primer
gcaggaaacagctatgaccaaacactaccacttcctttaatacaac

SEQ ID NO: 17 - DNA, 43 bp, Artificial Sequence
  Description of Artificial Sequence Synthetic primer
tgtaaaacgacggccagtatttttttgtttgttggggattggg

SEQ ID NO: 18 - DNA, 44 bp, Artificial Sequence
  Description of Artificial Sequence Synthetic primer
gcaggaaacagctatgaccaactacaccaatacaaccacatatc

SEQ ID NO: 19 - DNA, 37 bp, Artificial Sequence
  Description of Artificial Sequence Synthetic primer
tgtaaaacgacggccagtggttggttggttattagag

SEQ ID NO: 20 - DNA, 37 bp, Artificial Sequence
  Description of Artificial Sequence Synthetic primer
gcaggaaacagctatgaccccctctacccacctaaat

SEQ ID NO: 21 - DNA, 45 bp, Artificial Sequence
  Description of Artificial Sequence Synthetic primer
gtgtaaaacgacggccagtgttttattgtattagatttaagggaa

SEQ ID NO: 22 - DNA, 47 bp, Artificial Sequence
  Description of Artificial Sequence Synthetic primer
gcaggaaacagctatgaccctattaaataaaaaaaaaccccccaaac

SEQ ID NO: 23 - DNA, 43 bp, Artificial Sequence
  Description of Artificial Sequence Synthetic primer
gtgtaaaacgacggccagttttttttaggagtgaaggaggtta

SEQ ID NO: 24 - DNA, 43 bp, Artificial Sequence
  Description of Artificial Sequence Synthetic primer
gcaggaaacagctatgacccccaaaaaaaacaaaataaaaatc

SEQ ID NO: 25 - DNA, 37 bp, Artificial Sequence
  Description of Artificial Sequence Synthetic primer
tgtaaaacgacggccagttttaggtttttttttttgg

SEQ ID NO: 26 - DNA, 42 bp, Artificial Sequence
  Description of Artificial Sequence Synthetic primer
gcaggaaacagctatgaccctaaaaaataaaactccaaaaac

SEQ ID NO: 27 - DNA, 39 bp, Artificial Sequence
  Description of Artificial Sequence Synthetic primer
tgtaaaacgacggccagttaggttttttaggaaggagag

SEQ ID NO: 28 - DNA, 40 bp, Artificial Sequence
  Description of Artificial Sequence Synthetic primer
gcaggaaacagctatgaccctaaaaccccaactacctaaa

SEQ ID NO: 29 - DNA, 43 bp, Artificial Sequence
  Description of Artificial Sequence Synthetic primer
tgtaaaacgacggccagtgtttttagatagtaaagtttatttt

SEQ ID NO: 30 - DNA, 44 bp, Artificial Sequence
  Description of Artificial Sequence Synthetic primer
gcaggaaacagctatgacctacttataaaaaaactaaaatatcc

SEQ ID NO: 31 - DNA, 39 bp, Artificial Sequence
  Description of Artificial Sequence Synthetic primer
tgtaaaacgacggccagtgtttgtagggtttttattggt

SEQ ID NO: 32 - DNA, 44 bp, Artificial Sequence
  Description of Artificial Sequence Synthetic primer
gcaggaaacagctatgaccccctaactaaaaaaacaaaaactaa

SEQ ID NO: 33 - DNA, 44 bp, Artificial Sequence
  Description of Artificial Sequence Synthetic primer
tgtaaaacgacggccagtttttagtttaattttttatgatttag

SEQ ID NO: 34 - DNA, 42 bp, Artificial Sequence
  Description of Artificial Sequence Synthetic primer
gcaggaaacagctatgacctctaaatcctcctcaaaaaaaaa

SEQ ID NO: 35 - DNA, 42 bp, Artificial Sequence
  Description of Artificial Sequence Synthetic primer
tgtaaaacgacggccagtttttgtttatttgtggtttagata

SEQ ID NO: 36 - DNA, 43 bp, Artificial Sequence
  Description of Artificial Sequence Synthetic primer
gcaggaaacagctatgaccaaaaaacctaaactcattaaacta